SEQUENCE LISTING (3 sequences)

SEQ ID NO: 1 - protein, 146 aa, homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 2 - protein, 147 aa, rattus norvegicus
AVPIHKVQDDTKTLIKTIVTRINDISHTQSVSARQRVTGLDFIPGLHPILSLSKMDQTLAVYQQILTSLPSQNVLQIAHDLENLRDLLHLLAFSKSCSLPQTRGLQKPESLDGVLEASLYSTEVVALSRLQGSLQDILQQLDLSPEC

SEQ ID NO: 3 - protein, 147 aa, artificial
  Artificial Sequence based on SEQ ID 1 (homo sapiens, Leptin)
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

(19) United States
(12) Patent Application Publication
(10) Pub. No.: US 2014/0018290 A1
(43) Pub. Date: Jan. 16, 2014

(54) LEPTIN DERIVATIVES

(71) Applicants: Janos Tibor Kodra, Koebenhavn Oe (DK); Kilian Waldemar Conde-Frieboes, Maelov (DK); Johan Fredrik Paulsson, Malmoe (SE); Kirsten Raun, Lyngby (DK)

(72) Inventors: Janos Tibor Kodra, Koebenhavn Oe (DK); Kilian Waldemar Conde-Frieboes, Maelov (DK); Johan Fredrik Paulsson, Malmoe (SE); Kirsten Raun, Lyngby (DK)

(73) Assignee: Novo Nordisk A/S, Bagsvaerd (DK)

(21) Appl. No.: 13/979,773

(22) PCT Filed: Jan. 24, 2012
(86) PCT No.: PCT/EP2012/051055

§ 371 Date: Oct. 2, 2013

Related U.S. Application Data
(60) Provisional application No. 61/437,895, filed on Jan. 31, 2011.

(30) Foreign Application Priority Data
Jan. 26, 2011 (EP) 11152160.5

(51) Int. Cl. C07K 14/575; A61K 38/22

(52) U.S. Cl. CPC C07K 14/5759; A61K 38/2264; A61K 38/22
USPC 514/5.3; 530/359; 514/5.8

(57) ABSTRACT

The invention relates to Leptin derivatives, compositions and therapeutic use there-of.

### FIELD OF THE INVENTION

The present invention relates to novel Leptin derivatives and aspects related thereto, such as compositions thereof and therapeutic use thereof.

### BACKGROUND OF THE INVENTION

Leptin is a 16 kDa protein hormone that plays a key role in regulating energy intake and energy expenditure, including appetite and metabolism. Leptin is secreted predominantly by white adipose tissue. Studies in mice have demonstrated homozygous mutations of the Leptin gene cause massive obesity and lead to hyperglycaemic conditions in the ob/ob mice. Leptin administration decreases food intake and body weight in the ob/ob mouse model and corrects obesity-related metabolic and endocrine defects. Leptin is therefore a candidate for treatment of obesity. However obese humans are often characterized from being Leptin resistant and this have so far limited the use of Leptin as an anti-obesity agent.

Accordingly, a Leptin derivative, which in itself has improved in vivo potency and/or which in a combination therapy with further anti-obesity agent(s) can be dosed less frequently than human Leptin, is desirable in order to treat obesity.

### SUMMARY OF THE INVENTION

In one aspect the invention relates to a compound of the general formula Z—Y—X-Leptin compound, wherein
Z is an acyl group containing 12-22 carbon atoms and comprising a C-terminal carboxylic acid or a C-terminal tetrazole group;
Y is a spacer selected from the group consisting of a bond,

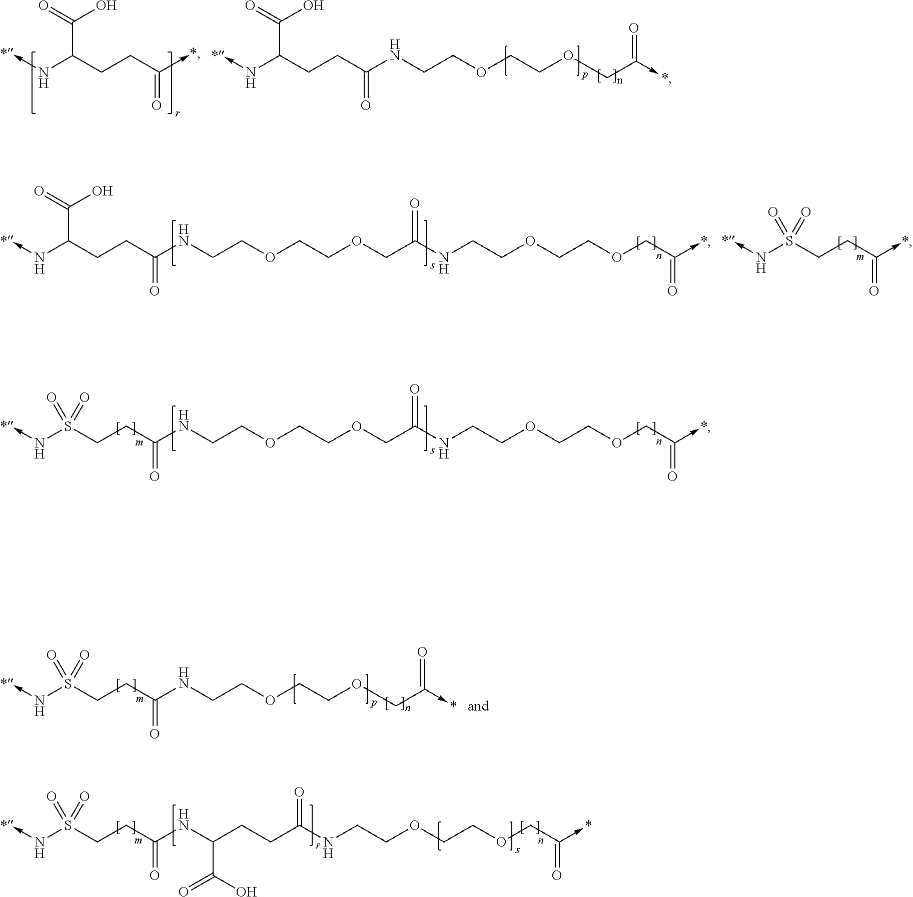

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23; X is the attachment anchor to the Leptin compound and is

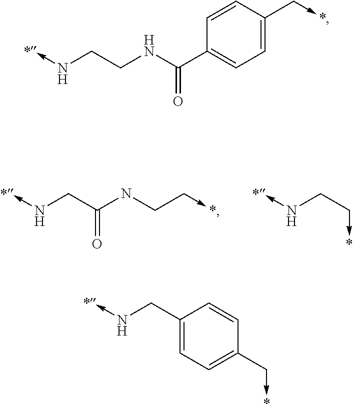

or
wherein “*” indicates the point of a moiety which is oriented towards the Leptin compound and “*″” indicates the point of a moiety which is oriented towards Z;
or a pharmaceutical salt, amide or ester thereof.

In one aspect Y is a spacer selected from the group consisting of a bond,

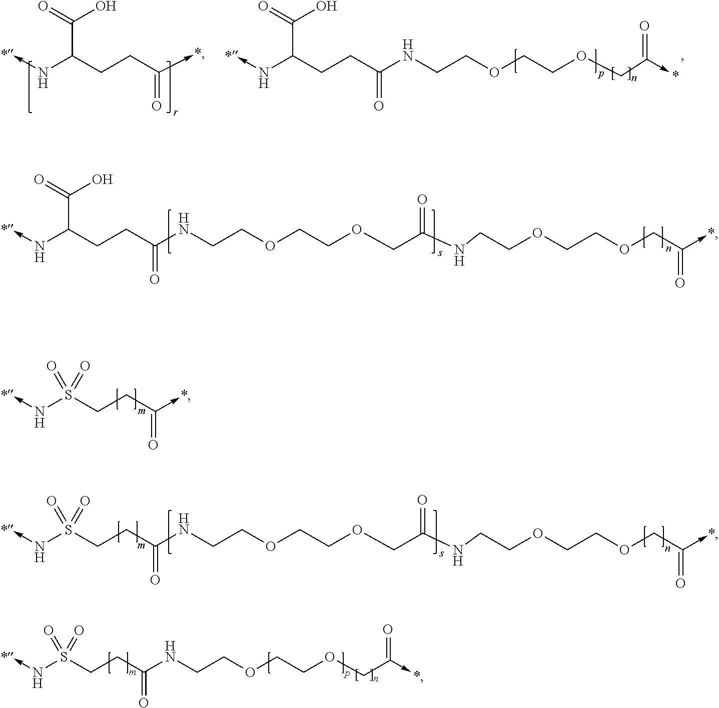

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23
and

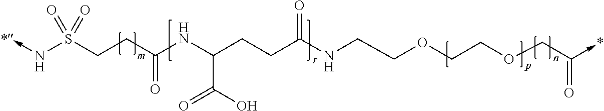

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2 or 3.
X is the attachment anchor to the Leptin compound and is

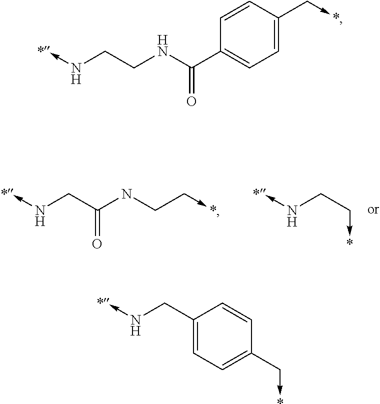

wherein “*” indicates the point of a moiety which is oriented towards the Leptin compound and “*″” indicates the point of a moiety which is oriented towards Z;
or a pharmaceutical salt, amide or ester thereof.

In one aspect the invention relates to a composition comprising a compound as defined herein and one or more pharmaceutical excipients, and optionally one or more further anti-obesity agents and/or anti-diabetes agents, such as pramlintide.

In one aspect the invention relates to a compound as defined herein for use in medicine. In one aspect the invention relates to a compound as described herein for the treatment of obesity, diabetes or lipodystrophy.

In one aspect the invention relates to a compound as described herein for the treatment of Type 2 Diabetes Mellitus.

In one aspect the invention relates to a compound as described herein for the treatment of complications and symptoms related to Type 2 Diabetes Mellitus.

In one aspect the invention relates to a compound as described herein for the treatment of complications and symptoms related to Type 2 Diabetes Mellitus, such as insulin resistance, hyperglycemia, hypertriglyceridemia and/or hepatic steatosis.

In one aspect the invention relates to a compound as described herein for the treatment of Type 1 Diabetes Mellitus.

In one aspect the invention relates to a compound as described herein for the treatment of complications and symptoms related to Type 1 Diabetes Mellitus.

In one aspect the invention relates to a compound as described herein for the treatment of complications and symptoms related to Type 1 Diabetes Mellitus, such as hyperglycemia.

In one aspect the invention relates to a compound as described herein for the treatment of congenital Leptin deficiency.

In one aspect the invention relates to a compound as described herein for the treatment of complications and symptoms related to congenital Leptin deficiency.

In one aspect the invention relates to a compound as described herein for the treatment of complications and symptoms related to congenital Leptin deficiency, due to gene mutations leading to insufficient levels of systemic Leptin.

In one aspect the invention relates to a compound as described herein for the treatment congenital lipoatrophy and/or lipodystrophy.

In one aspect the invention relates to a compound as described herein for the treatment of complications and symptoms related to congenital lipoatrophy and/or lipodystrophy.

In one aspect the invention relates to a compound as described herein for the treatment of complications and symptoms related to congenital lipoatrophy and/or lipodystrophy, resulting from adipose tissue reduction or low levels of systemic Leptin.

In one aspect the invention relates to a compound as described herein for the treatment of complications and symptoms related to congenital lipoatrophy and/or lipodystrophy. insulin resistance, hyperglycemia, hypertriglyceridemia and/or hepatic steatosis.

In one aspect the invention relates to a compound as described herein for the treatment HIV-associated lipodystrophy.

In one aspect the invention relates to a compound as described herein for the treatment of complications and symptoms related to HIV-associated lipodystrophy.

In one aspect the invention relates to a compound as described herein for the treatment of complications and symptoms related to HIV-associated lipodystrophy, due to Leptin deficiencies.

In one aspect the invention relates to a compound as described herein for the treatment of complications and symptoms related to HIV-associated lipodystrophy, such as insulin resistance, metabolic syndrome, hyperlipedemia and/or abdominal obesity.

In one aspect the invention relates to a compound as described herein for the treatment common obesity and/or weight loss maintenance (prevention of yo-yo effect related to dieting).

In one aspect the invention relates to a compound as described herein for the treatment common obesity, wherein Leptin resistance is a complication or symptom.

In one aspect the invention relates to a compound as described herein for the treatment of complications and symptoms related to common obesity.

In one aspect the invention relates to a compound as described herein for the treatment of cessation and/or irregularities of menstrual cycle and side effects thereof, such as amenorrhea (primary and secondary).

In one aspect the invention relates to a compound as described herein for the treatment of complications and symptoms related to as amenorrhea (primary and secondary).

In one aspect the invention relates to a compound as described herein for the treatment of complications and symptoms related to as amenorrhea (primary and secondary), such as related to the cessation and/or irregularities of menstrual cycle.

In one aspect the invention relates to a compound as described herein for the treatment of irregular menstruation cycles, such as in Polycystic Ovarian Syndrome (PCOS).

In one aspect the invention relates to a compound as described herein for the treatment in Polycystic Ovarian Syndrome (PCOS).

In one aspect the invention relates to a compound as described herein for the treatment of complications and symptoms related to Polycystic Ovarian Syndrome (PCOS).

In one aspect the invention relates to a compound as described herein for the treatment of complications and symptoms related to Polycystic Ovarian Syndrome (PCOS), such as irregular menstruation cycles.

In one aspect the invention relates to a compound as described herein for the treatment of bone mass loss, such as in Osteoporosis.

In one aspect the invention relates to a compound as described herein for the treatment of Osteoporosis.

In one aspect the invention relates to a compound as described herein for the treatment of complications and symptoms related to Osteoporosis.

In one aspect the invention relates to a compound as described herein for the treatment of complications and symptoms related to Osteoporosis, such as bone mass loss and/or bone weakness.

In one aspect the invention relates to use of a compound as defined herein for the preparation of a medicament for the treatment of obesity, diabetes or lipodystrophy.

### BRIEF DESCRIPTION OF DRAWINGS

FIG. 1 shows the effect on body weight reduction in Leptin sensitive ob/ob mice after single injection of Leptin derivatives according to example 2 and 3 (Compound A and B) compared to unmodified human/rat Leptin after 6 days, MEAN±SEM, n=6-7

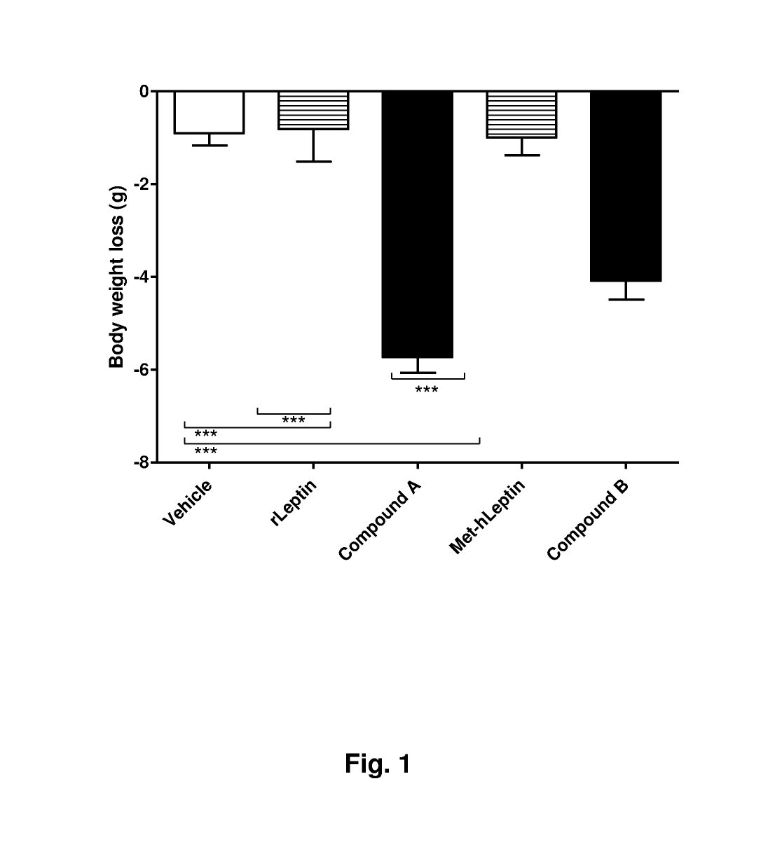

FIG. 2 shows the effect on body weight reduction in Leptin sensitive ob/ob mice after single injection of Leptin derivatives according to example 5 and 6 (Compound C and D) compared to unmodified human/rat Leptin after 6 days, MEAN±SEM, n=6-7

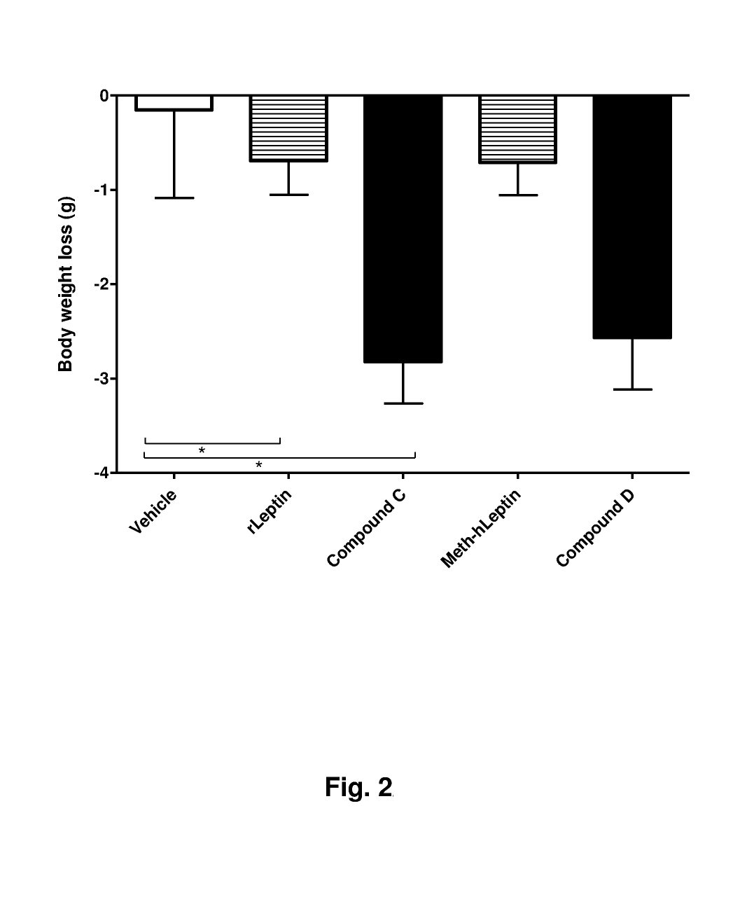

FIG. 3 shows the effect on blood glucose in Leptin sensitive ob/ob mice after single injection of Leptin derivatives according to example 2 and 3 (Compound A and B) compared to unmodified human/rat Leptin.

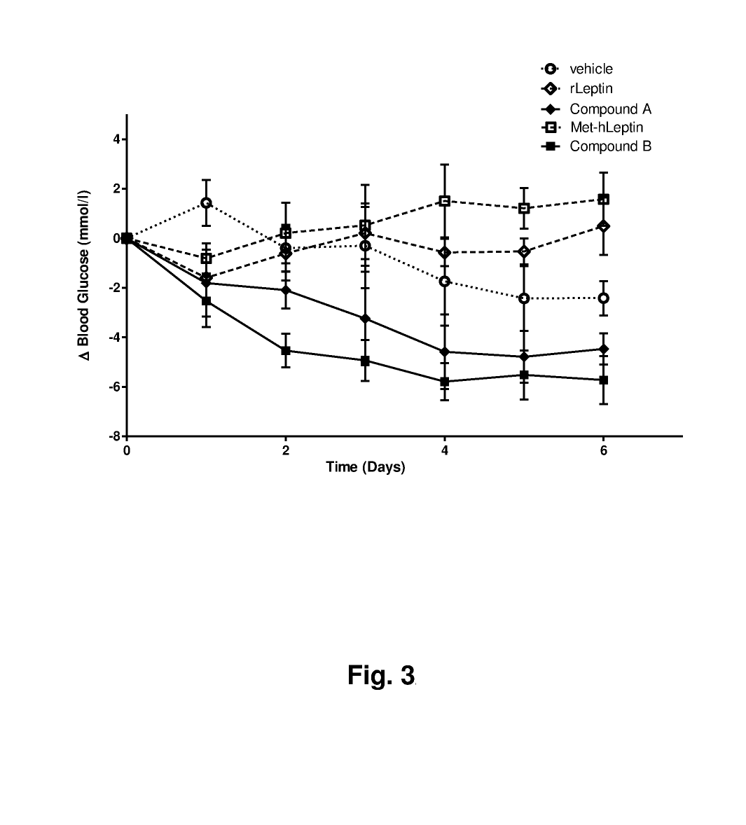

FIG. 4 shows the effect on blood glucose in Leptin sensitive ob/ob mice after single injection of Leptin derivatives according to example 5 and 6 (Compound C and D)compared to unmodified human/rat Leptin.

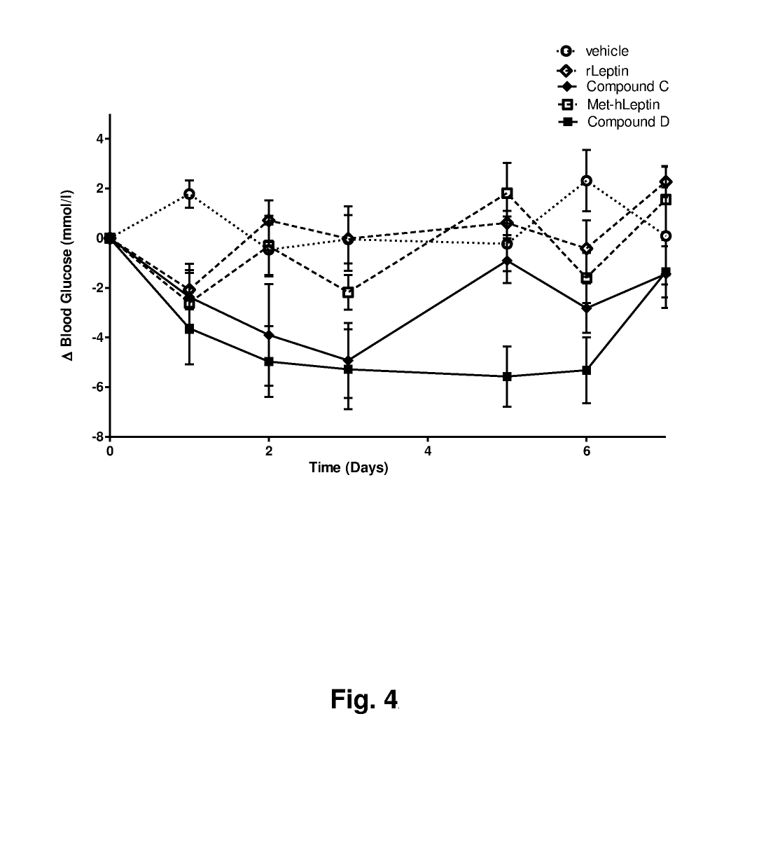

### DESCRIPTION OF THE INVENTION

In one aspect the present invention relates to a compounds of the general formula Z—Y—X-Leptin compound; Z is an acyl group; Y is a spacer as defined herein; and X is an attachment group as defined herein.

In one aspect the present invention relates to a compound of the general formula Z—Y—X-Leptin compound, wherein
Z is an acyl group containing 12-22 carbon atoms and comprising a C-terminal carboxylic acid or a C-terminal tetrazole group;
Y is a spacer selected from the group consisting of a bond,

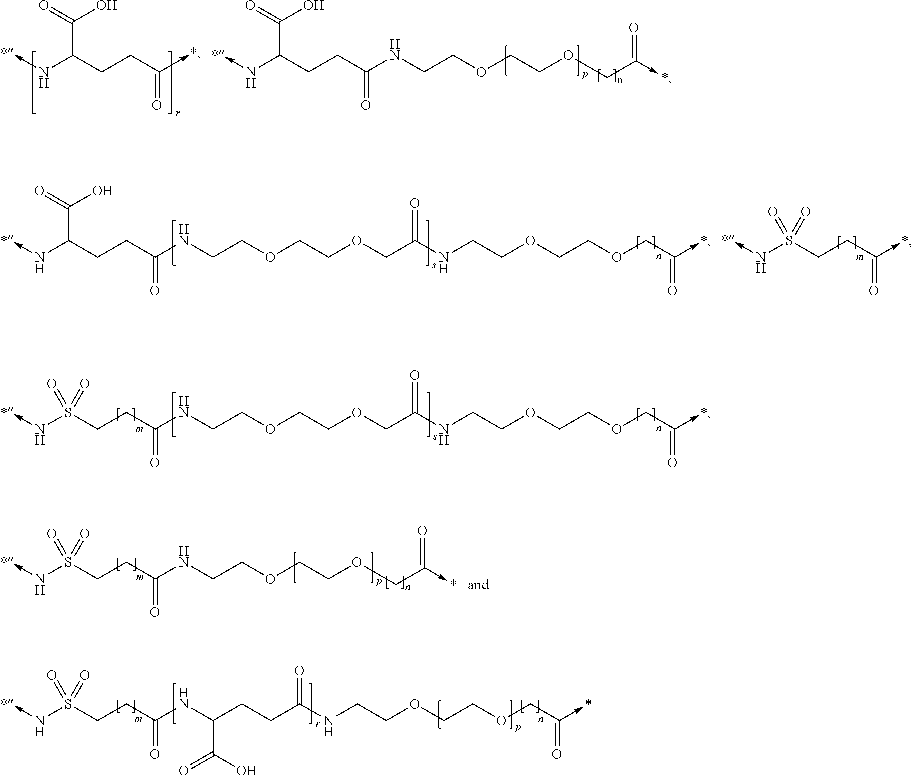

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23;
X is the attachment anchor to the Leptin compound and is

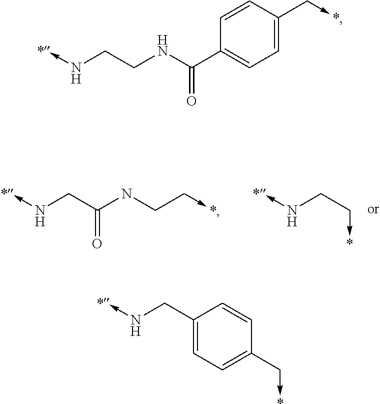

wherein “*” indicates the point of a moiety which is oriented towards the Leptin compound and “*″” indicates the point of a moiety which is oriented towards Z;
or a pharmaceutical salt, amide or ester thereof.

In one aspect Y is a spacer selected from the group consisting of a bond,

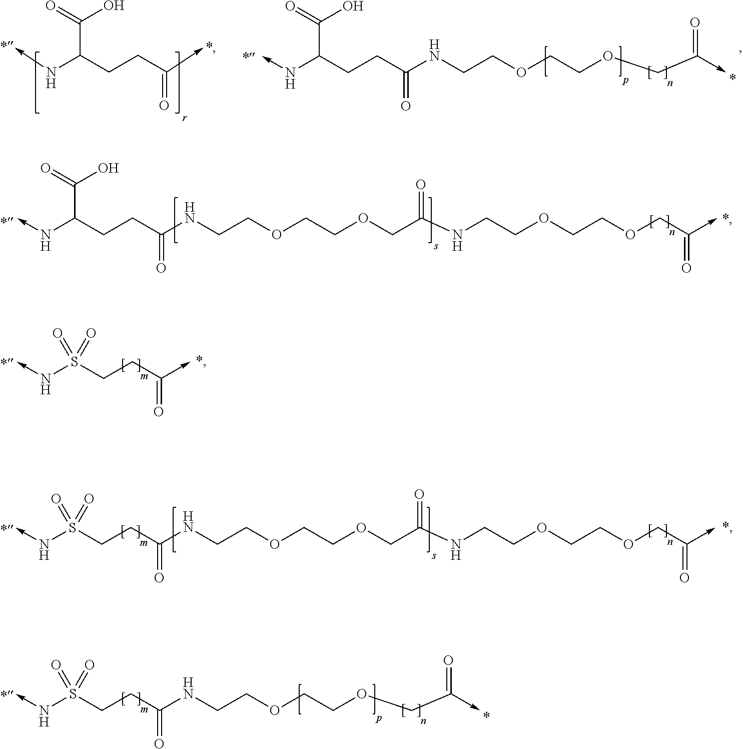

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23 and

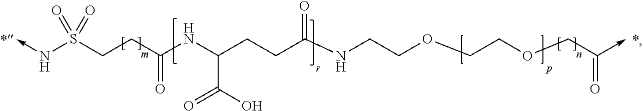

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2 or 3;
X is the attachment anchor to the Leptin compound and is

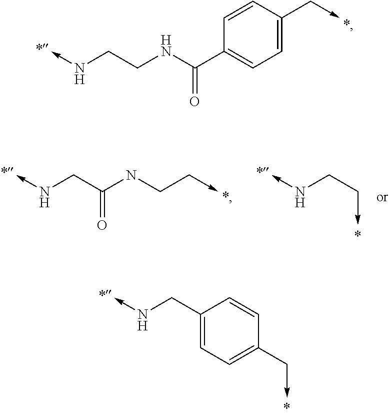

wherein “*” indicates the point of a moiety which is oriented towards the Leptin compound and “*″” indicates the point of a moiety which is oriented towards Z;
or a pharmaceutical salt, amide or ester thereof.

In one aspect the present invention relates to a compound of the general formula Z—Y—X-Leptin compound, wherein
Z is an acyl group containing 16-18 carbon atoms and comprising a C-terminal carboxylic acid or a C-terminal tetrazole group;
Y is a spacer selected from the group consisting of a bond,

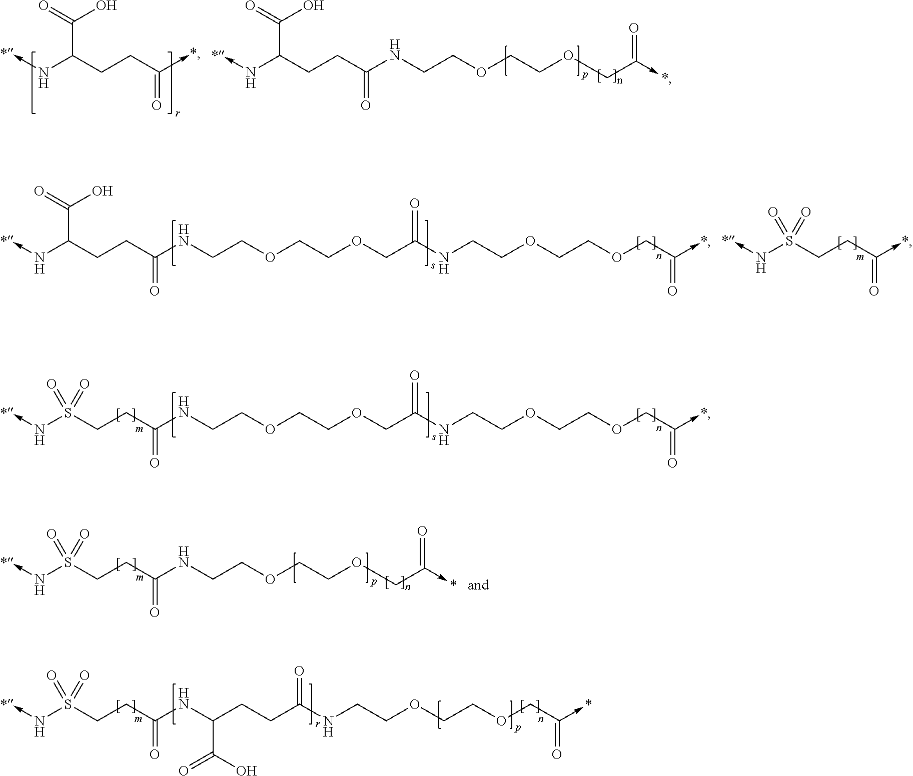

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23;
X is the attachment anchor to the Leptin compound and is

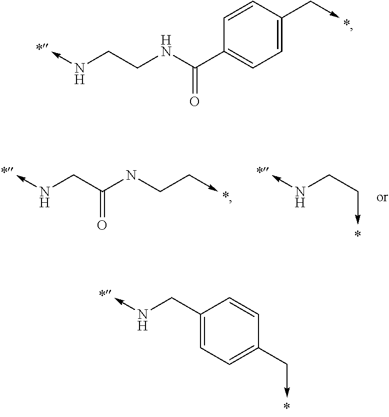

wherein “*” indicates the point of a moiety which is oriented towards the Leptin compound and “*″” indicates the point of a moiety which is oriented towards Z;
or a pharmaceutical salt, amide or ester thereof.

In one aspect Y is a spacer selected from the group consisting of a bond,

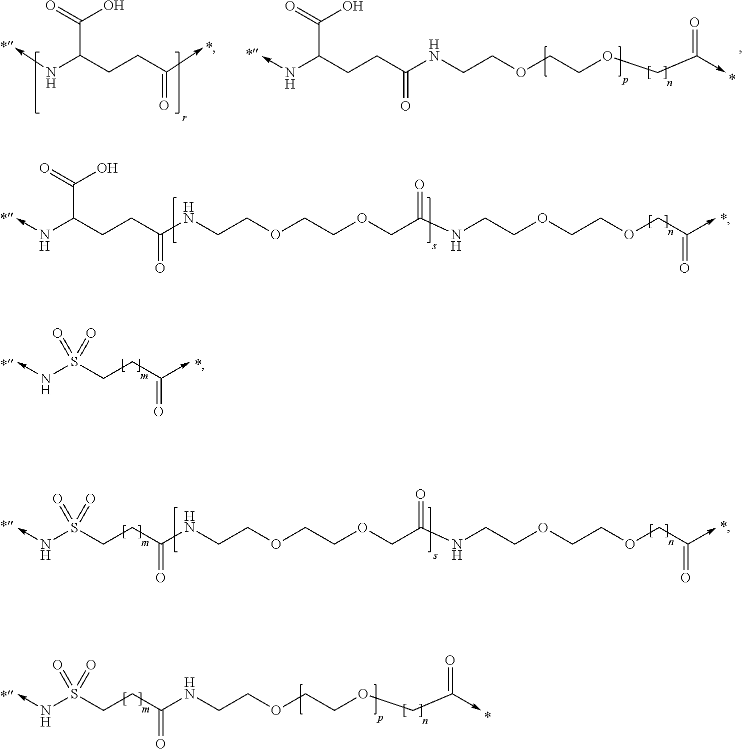

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23
and

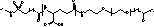

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2 or 3; X is the attachment anchor to the Leptin compound and is

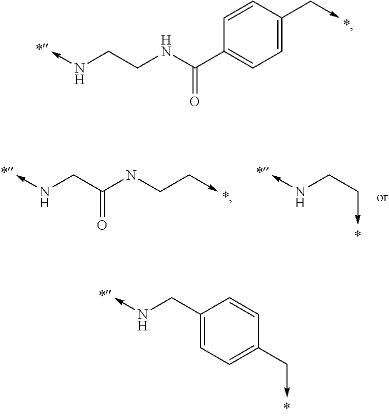

wherein “*” indicates the point of a moiety which is oriented towards the Leptin compound and “*″” indicates the point of a moiety which is oriented towards Z;
or a pharmaceutical salt, amide or ester thereof.

In one aspect said Z—Y—X-moiety is connected to an amino group present in an amino acid residue present in the Leptin compound or to the N terminal alpha-amino group in the Leptin compound. In one aspect a hydrogen has been removed from said amino group.

In one aspect the X moiety of said Z—Y—X moiety is attached to the Leptin compound by alkylation chemistry.

In one embodiment said Z—Y—X moiety of said Z—Y—X-Leptin compound is attached to the Leptin compound by alkylation chemistry.

In one aspect the compound as defined herein provides a Leptin of a wt Leptin.

In one aspect the present invention provides a Leptin of a wt Leptin analogue. In one aspect the compound as defined herein provides a Leptin derivative comprising a Z—Y—X moiety which is selectively attached to the N-terminus of the wt Leptin.

In one aspect the compound as defined herein provides a rat Leptin derivative comprising a Z—Y—X moiety which is selectively attached to the N-terminus of the wt rat Leptin.

In one aspect the compound as defined herein provides a human Leptin derivative comprising a Z—Y—X moiety which is selectively attached to the N-terminus of the wt human Leptin.

In one aspect the X moiety of said Z—Y—X moiety is attached to a Met-human Leptin by alkylation chemistry.

In one aspect the the compound as defined herein provides a Leptin derivative of Leptin.

In one aspect the compound as defined herein provides a Leptin derivative of Leptin and is selectively alkylated in the N-terminus.

In one aspect the the compound as defined herein provides a Leptin derivative of human Leptin (SEQ ID NO: 1).

In one aspect the compound as defined herein provides a Leptin derivative of human Leptin (SEQ ID NO: 1) and is selectively alkylated in the N-terminus.

In one aspect the the compound as defined herein provides a Leptin derivative of rat Leptin (SEQ ID NO: 2).

In one aspect the compound as defined herein provides a Leptin derivative of rat Leptin (SEQ ID NO: 2) and is selectively alkylated in the N-terminus.

In one aspect the compound as defined herein provides a Leptin derivative of of Met-human Leptin (SEQ ID NO: 3).

In one aspect the compound as defined herein provides a Leptin derivative of of Met-human Leptin (SEQ ID NO: 3) and is selectively alkylated in the N-terminus.

In one aspect the compound as defined herein provides a Leptin derivative which is biologically active.

In one aspect said Leptin compound is an analogue of Leptin, such as an analogue of rat or human Leptin. In one aspect said Leptin compound has 90%, such as 95% or 98% sequence identity, to human Leptin.

In one aspect the Leptin compound is derived from a mammal, such as a human, pig, rat or mouse or such as human or rat. In one aspect the Leptin compound is human Leptin as defined by VPIQKVQDDTKTLIKTIVTRINDISHTQSVSSKQKVTGLDFIPGLHPILTL-SKMD-QTLAVYQQILT SMPSRNVIQISNDLENLRDLLHVLAFSKSCHLPWASGLETLDSLG-GVLEAS-GYSTEVVALSRLQGSLQDMLWQLDLSPGC (SEQ ID NO: 1). The terms “human Leptin” and “hLeptin” are used interchangeably herein to describe SEQ ID NO: 1. In one aspect the Leptin compound is rat Leptin as defined by AVPIHKVQDDTKTLIK TIVTRIN-DISHTQSVSARQRVT-GLDFIPGLHPI LSLSKMDQTLAVYQQILTSLPSQNVLQIAHDLENL-RDLLHLLAFSKSCSLPQ-TRGLQKPESLD GVLEASLYSTEWALSRLQGSLQDILQQLDL SPEC (SEQ ID NO: 2). The terms “rat Leptin” and “rLeptin” are used interchangeably herein to describe SEQ ID NO: 2.

The terms “Met-human Leptin” and “Met-hLeptin” are used interchangeably herein to describe SEQ ID NO: 3. In one aspect the Leptin compound is Met-human Leptin as defined by MVPIQKVQDDTKTLIKTIVTRINDISHTQSVSSKQKVTGLDFIPGLHPILTLSKMD-QTLAVYQQILT SMPSRNVIQISNDLENLRDLLHVLAFSKSCHLPWASGLETLDSLGGVLEAS-GYSTEVVALSRLQGSLQDMLWQLDLSPGC (SEQ ID NO: 3).

The term “Leptin” as used herein refers to the wild type (wt) variant of mammalian Leptin, if not indicated differently. The term “wt” or “native” Leptin or “wt of Leptin” as used herein refers to a wt (wild type) peptide, or a compound, which is a variant of a mammalian Leptin. SEQ ID NO: 1 as included in the Sequence list is an example of a rat “wt Leptin” may be designated “rat Leptin” and SEQ ID NO: 2 as included in the Sequence list is an example of a human “wt Leptin” may be designated “human Leptin”. The peptide having the sequence of SEQ ID NO: 1 may also be designated “native” rat Leptin or “native” rLeptin. The peptide having the sequence of SEQ ID NO: 2 may also be designated “native” or “wt” human Leptin or “native” or “wt” hLeptin.

The term “Leptin compound” as used herein refers to mammalian Leptin or Met-human Leptin and therefore includes wt variants of Leptin and Leptin analogues as defined herein. The term “compound as defined herein” or “compound as described herein” as used herein designated “Leptin derivatives” and/or modified “Leptin compounds” as defined in the description and/or claims.

In one aspect the Leptin compound is an analogue of Leptin, such as an analogue of rat or human Leptin. In one aspect the term “analogue” of a peptide is intended to mean said peptide wherein one or more amino acid residues have been substituted, deleted or inserted. In one aspect the term “amino acid residue” is intended to mean said an amino acid from which, formally, a hydroxy group has been removed from a carboxy group and/or from which, formally, a hydrogen atom has been removed from an amino group.

The term “Leptin analogue” as used herein means a modified human Leptin wherein one or more amino acid residues of the wt Leptin have been substituted by other amino acid residues and/or wherein one or more amino acid residues have been deleted from the Leptin and/or wherein one or more amino acid residues have been added and/or inserted to the Leptin.

Herein the terms “alpha-ala-rLeptin” and “alpha-rLeptin” are used interchangeably herein to describe that the substitution is at the N-terminal amino group.

Herein the terms “alpha-ala-rLeptin” and “alpha-rLeptin” are used interchangeably herein to describe that the substitution is at the N-terminal alpha-amino group of an Alanine.

In one aspect the Leptin compound has 90%, such as 95% or 98% sequence identity, to human Leptin. In one aspect “sequence identity” is determined over the entire peptide, wherein two peptide analogues are aligned and the sequence identity of the first analogue relative to the second analogue is given by the number of aligned identical residues minus the number of different residues divided by the total number of residues in the first analogue. Accordingly, the sequence identity of the peptide AAEAA relative to the peptide AAAAA is (5-1)/5.

In one aspect a Leptin analogue comprises less than 10 amino acid modifications (substitutions, deletions, additions (including insertions) and any combination thereof) relative to human Leptin, alternatively less than 9, 8, 7, 6, 5, 4, 3, 2 or 1 modification relative to human Leptin.

The term “Leptin derivative” or “protracted Leptin” as used herein means a chemically modified Leptin compound or a Leptin analogue, wherein the modification(s) are in the form of attachment of side chains. Side chains according to the present invention include, but are not limited to Z—Y—X moieties as defined in the description.

The term “Z—Y—X Leptin Compound” as used herein may also be designated by, and thus include the definition of, the term “Leptin derivative”.

The term “Met-human Leptin” or” Met-hLeptin” as used herein refers to a human Leptin analogue that comprises a Methionine amino acid in the N-terminus. This includes Met-human Leptin which is derived by expression in E. Coli. The peptide having the sequence of SEQ ID NO: 3 is an example of such a Met-human Leptin and may also be designated “Met-human Leptin” or “Met-hLeptin”.

In one aspect the Z—Y—X-moiety is connected to an amino group, which is the N-terminal amino group or present in an amino acid residue present in the Leptin compound.

In one aspect Z is an acyl group containing 12-22 carbon atoms and comprising a C-terminal carboxylic acid or a C-terminal tetrazole group. In one aspect Z comprises an acyl group. In one aspect Z is an acyl group. In one aspect Z comprises 12-22 carbon atoms. In one aspect Z comprises a distal carboxylic acid group. In one aspect Z comprises a distal tetrazole group. In one aspect Z comprises a fatty acid or fatty diacid. In one aspect Z is a fatty acid or fatty diacid. In one aspect Z comprises an alpha and omega carboxy group. In one aspect Z is a fatty acid or fatty diacid with 12-22 carbon atoms, such as 16, 18 or 20 carbon atoms. In one aspect Z is

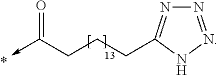

In one aspect the spacer, Y, is selected from the group consisting a bond,

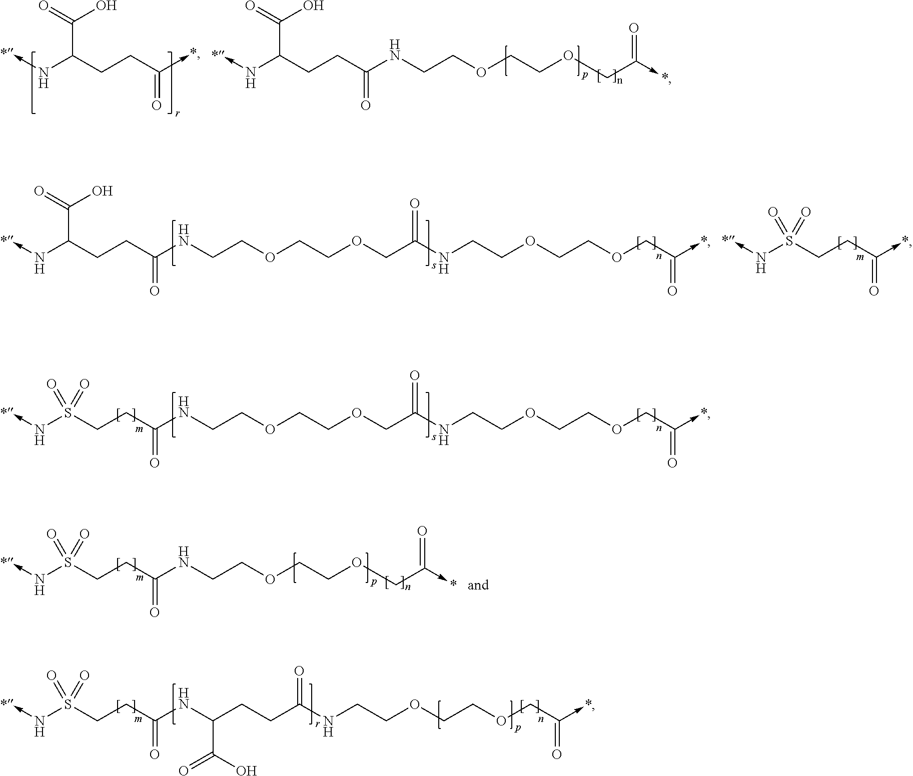

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23.

In one aspect Y is a spacer selected from the group consisting of a bond,

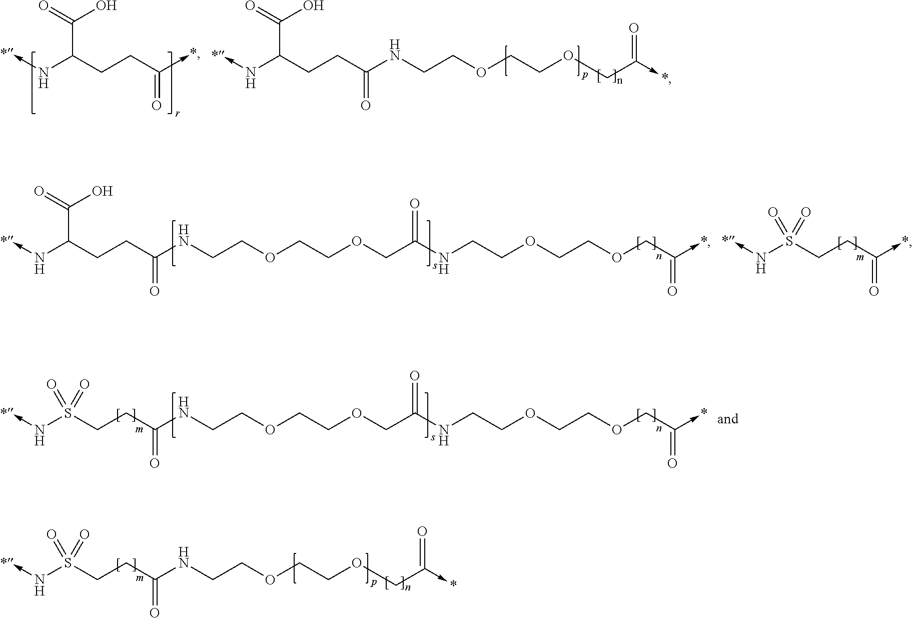

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23
and

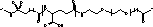

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2 or 3.

In one aspect Y is a bond. In one aspect Y is

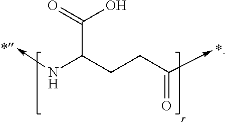

In one aspect Y is

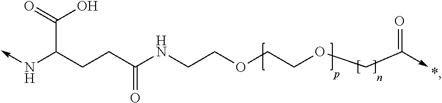

In one aspect Y is

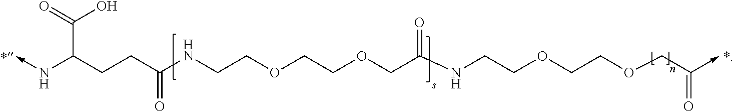

In one aspect Y is

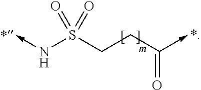

In one aspect Y is

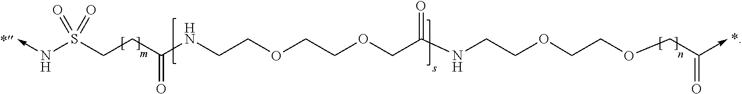

In one aspect Y is

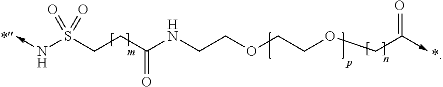

In one aspect Y is

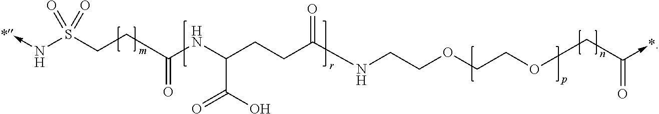

In one aspect m is 0, 1, 2, 3, 4, 5 or 6. In one aspect n is 1, 2 or 3. In one aspect s is 0, 1, 2 or 3. In one aspect p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23;

In one aspect m is 0, 1, 2, 3, 4, 5 or 6. In one aspect n is 1, 2 or 3. In one aspect s is 0, 1, 2 or 3. In one aspect p is 1, 2, 3, 4, 5, 6, 7, 8, 9, 10, 11, 12, 13, 14, 15, 16, 17, 18, 19, 20, 21, 22 or 23; r is 1, 2,or 3.In one aspect m is 0,1 or 2, r is 1 or 2, p is 1, n is 1.

In one aspect m is 0 or 1; r is 1 or 2; p is 1; n is 0 or 1.

In one aspect m is 0 or 2; r is 1 or 2; p is 1; n is 0 or 1.

In one aspect m is 0 or 1; r is 1, p is 1; n is 0 or 1.

In one aspect m is 0 or 2; r is 2; p is 1; n is 1.

In one aspect m is 1; n is 1; s is 1 or 2.

In one aspect m is 0 or 1; n is 0 or 1; s is 1.

In one aspect m is 0 or 1; n is 0 or 1; s is 2.

Y is a spacer selected from the group consisting of a bond,

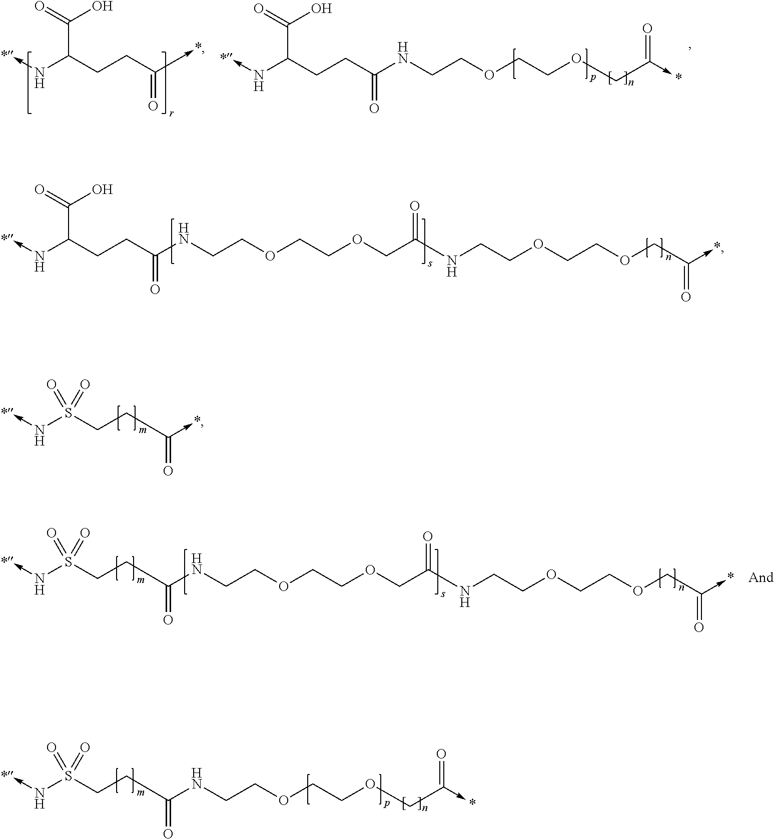

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23
and

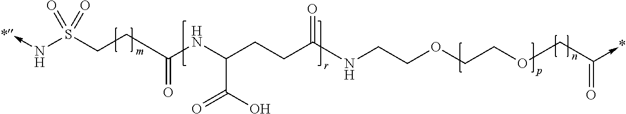

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2 or 3.

In one aspect X is

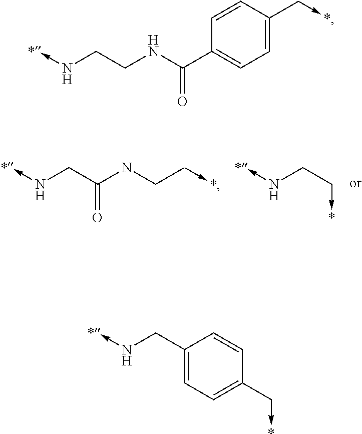

In one aspect, in the formulas herein “→” indicates the atom from which a bond from a first moiety to a second moiety, such as from Y to X, and herein “*” indicates the point of a moiety which is oriented towards the Leptin compound and “*″” indicates the point of a moiety which is oriented towards Z, i.e. for formulas representing X “*” indicates the point of attachment of X to the Leptin compound and “*″” indicates the point of attachment of X to Y.

In one aspect X is the attachment anchor to the Leptin compound generated from an aldehyde which is either free or formed by by deprotection of an acetal, such as

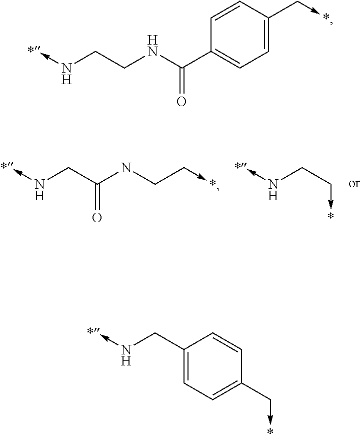

In one aspect the a-carbonyl end of Z is connected to the amino end of Y via an amide bond and the carbonyl end of Y is connected to the amino end of X via an amide bond. In one aspect the a-carbonyl end of Z is connected to the amino end of X via an amide bond.

In one aspect the Z—Y—X-moiety is

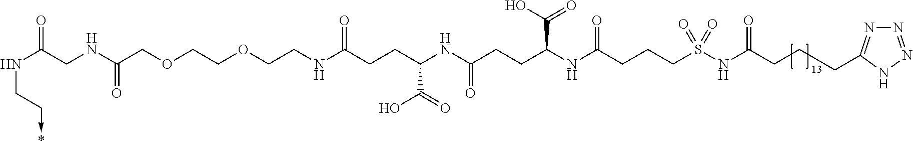

In one aspect compounds of the the Z—Y—X-moiety is.

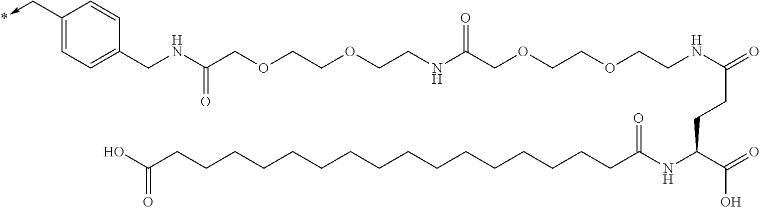

In one aspect compounds of the invention comprise all stereoisomers of the Z—Y—X-moiety.

A compound according to the invention is

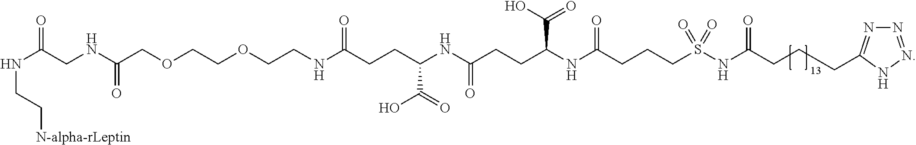

### (Compound A).

A compound according to the invention is

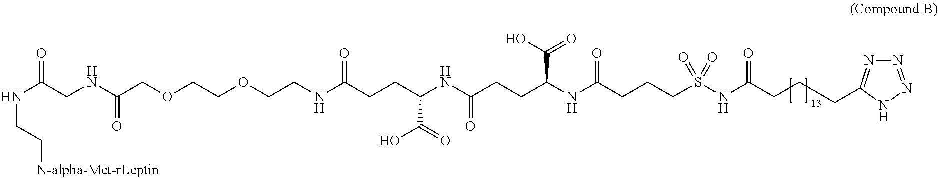

A compound according to the invention is

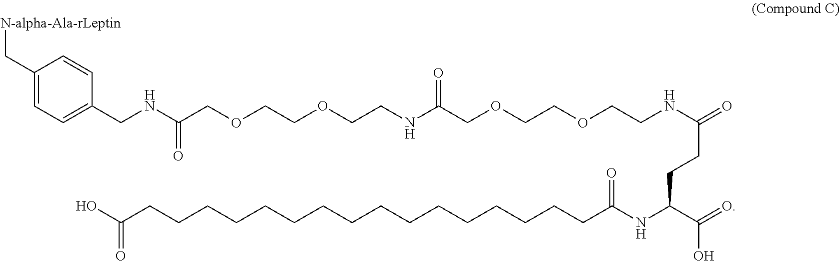

A compound according to the invention is

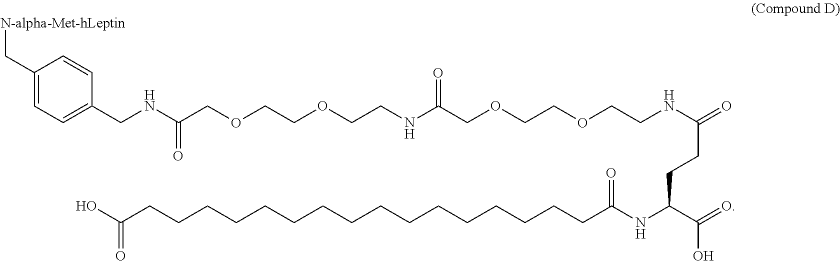

### Method of Preparation

The Leptin compounds (i.e. wt or native Leptin) of this invention can be prepared in a manner known per se. Rat and human Leptin are commercially available (RayBiotech, Inc., Norcross, Ga., USA). Met-human Leptin was made as described below. Another strategy could be first to prepare the Leptin compound. The Leptin compound can be expressed using method known for the person skilled in the art, see for example U.S. Pat. No. 6,025,324 and U.S. Pat. No. 6,025,325. The Z—Y—X-moiety can be made using method known for the person skilled in the art, such as described in European patent WO11015649. A non-limiting example of such a method is found on page 76 of WO2011/015649.

### Pharmacological Effects

In one aspect the invention relates to the use of a compound as defined herein for use in medicine. In one aspect the invention relates to the use of a compound as defined herein for the treatment of obesity, diabetes or lipodystrophy. In one aspect the invention relates to the use of a compound as defined herein for the preparation of a medicament for the treatment of obesity, diabetes or lipodystrophy. In one aspect the invention relates to a method of treatment of obesity or diabetes, wherein a compound as defined herein is administered to a patient in need thereof.

Leptin is an important hormone normal regulation of reproduction. In one aspect the invention relates to the use of a compound as defined herein for the treatment of delayed puberty, amenorrhea or polycystic ovarian syndrome.

The pharmacological effects of the compounds of this invention are beneficial for the treatment of obesity, especially since they have a prolonged action. In one aspect the invention relates to the use of a compound as defined herein, wherein said compound is administered to a subject in need thereof once daily or less frequently, such as once-weekly.

In one aspect the compounds of this invention shall have a sufficient effect on food intake. The effect on food intake can be determined by the method described in Assay (I) herein.

In one aspect the compounds of this invention shall have a sufficient effect on body weight. The effect on body weight can be determined by the method described in Assay (I) herein.

Pharmaceutical formulations containing a compound of this invention can, for example, be used for reduction of food intake and reduction of body weight. Hence, pharmacological treatment with a compound of this invention may be suitable for the treatment or prevention of obesity.

### Pharmaceutical Compositions

In one aspect the invention relates to a composition comprising a compound as defined herein and one or more pharmaceutical excipients. In one aspect said composition further comprises one or more further anti-obesity agents and/or anti-diabetes agents and optionally one or more pharmaceutical excipients. In one aspect one of said anti-obesity agents and/or anti-diabetes agents is pramlintide.

In one aspect the invention relates to a composition comprising a compound as defined herein and one or more pharmaceutical excipients.

In one aspect according to this invention a therapeutically effective amount of a compound of this invention is administered to a subject (for example, patient or animal) who would benefit from such a treatment. The treatment could, for example, be obesity. The dosage ranges for the administration of the compound of this invention are those large enough to produce the desired effect.

In one aspect this invention provides a compound of this invention in a unit dosage form for administration to patients. As used herein, “unit dosage form” refers to a composition intended for a single administration to treat a subject suffering from a disease or medical condition. Each unit dosage form typically comprises each of the compounds of this invention plus pharmaceutically acceptable excipients. Examples of unit dosage forms are individual tablets, individual capsules, bulk powders, liquid solutions, suppositories, emulsions or suspensions. Treatment of the disease or condition may require periodic administration of unit dosage forms, for example: one unit dosage form two or more times a day, one with each meal, one every four hours or other interval, or only one per day. Formulations for injection may be presented in unit dosage form, for example, in ampoules or in multidose containers.

The unit dosage form of the invention contains a therapeutically effective dose of a compound of this invention. In one aspect administration of the unit dosage form results in a proper level of a compound of this invention in the mammal.

Although the particular dose will depend on the molecular structure and chemical properties of the particular compound of this invention, those of skill in the pharmacology art will understand from the disclosure herein that appropriate doses can be determined using routine techniques. For example, a dose or formulation of a compound of this invention with no or only minimally eliciting an undesired side-effect in the mammal can be determined in a variety of ways. As used in this context, “an increased level” can refer to an increase to a predetermined level (for example, a designated threshold level of the side effect). One method for making such determination involves conducting dose-response assays by (a) administering a plurality of different doses (or formulations) of a compound of this invention to test mammals; and (b) measuring the effect of each dose or formulation and measuring the effect of each dose on the side-effect, thereby creating dose-response data for the desired effect and the side-effect; and, (ii) determining from the dose-response data a dose of the a compound of this invention formulation that gives the desired effect but does not elicit the side-effect.

The amount of a compound of this invention administered to an animal to achieve a desired level or concentration of the compound of this invention will depend on a number of factors well known to practitioners, such as compound half-life (for example, serum half-life), and the frequency and mode of administration. Other ranges of a compound of this invention will be apparent to the skilled practitioner based on data from initial dose-response curves and other data that can be obtained by routine methods.

The invention also provides a composition containing a compound of this invention combined with one or more pharmaceutically acceptable excipients.

In one aspect the composition comprises a buffer, such as a phosphate buffer or Na₂HPO₄.

In one aspect the composition comprises glycerol.

In one aspect the composition comprises an isotonicity agent, such as NaCl.

In one aspect pH of the composition is in the range of pH 3-10, such as pH 7.5.

In one aspect the composition comprises Na₂HPO₄, glycerol and NaCl. In one aspect the composition comprises 15 mM Na₂HPO₄, 7.5% (v/v) glycerol, 125 mM NaCl, pH 7.5.

A compound of this invention can be directly administered to the subject to be treated. Administration is optionally under sterile conditions. However, while it is possible for a compound of this invention to be administered alone, it is often preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious to the subject. Therapeutic formulations can be prepared by any methods well known in the art of pharmacy.

A compound of this invention may be administered by parenteral (for example, intra-muscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. Often, the administration will be parenterally (for example, intravenous).

If desired (for example, to maintain a particular plasma concentration) a compound of this invention can be administered to patients in the form of controlled delivery formulations. A variety of suitable controlled delivery systems are known, including forms suitable for parenteral, and other routes of administration. Excipients employed in the manufacture of drug delivery systems are described in various publications known to those skilled in the art. This publication also presents general chapters and specific tests to determine the drug release capabilities of extended-release and delayed-release tablets and capsules. In one aspect of the invention, a compound of this invention is administered in conjunction with a program of exercise, to enhance exercise-mediated breakdown of triglycerides in a subject.

The pharmaceutical compositions may be in the form of a sterile injectable aqueous or oleagenous suspension. This suspension may be formulated according to the known art using those suitable dispersing or wetting agents and suspending agents. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, and the severity of the particular condition. In some embodiments, daily or weekly administration of a compound of this invention is contemplated.

Herein, the expression that an acylated derivative according to the present invention has a “prolonged action” means that the T½ thereof is at least 50%, preferably at least 100%, and more preferred at least 500%, longer than the T½ of the corresponding non-derivatised Leptin.

Herein, the expression that an alkylated derivative according to the present invention has a “prolonged action” means that the T½ thereof is at least 50%, preferably at least 100%, and more preferred at least 500%, longer than the T½ of the corresponding non-derivatised Leptin.

Herein, the expression that an alkylated derivative according to the present invention has a “prolonged effect” means that the pharmacodynamic effects thereof is increased relative to corresponding non-derivatised Leptin. In one aspect the increase is at least 50%, preferably at least 100%, and more preferred at least 500%, longer the pharmacodynamics effects corresponding non-derivatised Leptin.

Herein the expression “pharmacodynamic effect” is the biochemical and physiological effects on the body. In one embodiment the “pharmacodynamic effect” refers to the inhibitory effects of Leptin on body weight, food intake or blood glucose.

Herein, “therapeutically effective amount” refers to a predetermined amount of an agent calculated to elicit the biological or medical response of a tissue, system, animal or human that is being sought by the researcher, veterinarian, physician or other clinician, for example, an amount sufficient to stimulate, prevent, hinder, retard or reverse the progression of a disease or any other undesirable symptoms to achieve a desired therapeutic effect.

Herein, “pharmaceutically acceptable carrier” or “pharmaceutically acceptable excipient” refers to a carrier that does not cause an adverse physical reaction upon administration and one in which a therapeutic agent is sufficiently soluble to deliver a therapeutically effective amount. Examples of excipients include buffered water, physiological saline, phosphate buffered saline (PBS), dextrose solution, Hank's solution and inert diluents, such as calcium carbonate, sodium carbonate, lactose, calcium phosphate or sodium phosphate.

Herein, “mammal” has its usual meaning and includes primates (for example, humans and non-human primates), experimental animals (for example, rodents such as mice and rats), farm animals (such as cows, hogs, sheep and horses), and domestic animals (such as dogs and cats).

Herein, the terms “treatment” or “treating” of a condition and/or a disease in a mammal, means (i) preventing the condition or disease, that is, avoiding any clinical symptoms of the disease; (ii) inhibiting the condition or disease, that is, arresting the development or progression of clinical symptoms; and/or (iii) relieving the condition or disease, that is, causing the regression of clinical symptoms.

### Embodiments of the Invention

1. A compound of the general formula Z—Y—X-Leptin compound, wherein
Z is an acyl group containing 12-22 carbon atoms and comprises a C-terminal carboxylic acid or a C-terminal tetrazole group;
Y is a spacer selected from the group consisting of a bond,

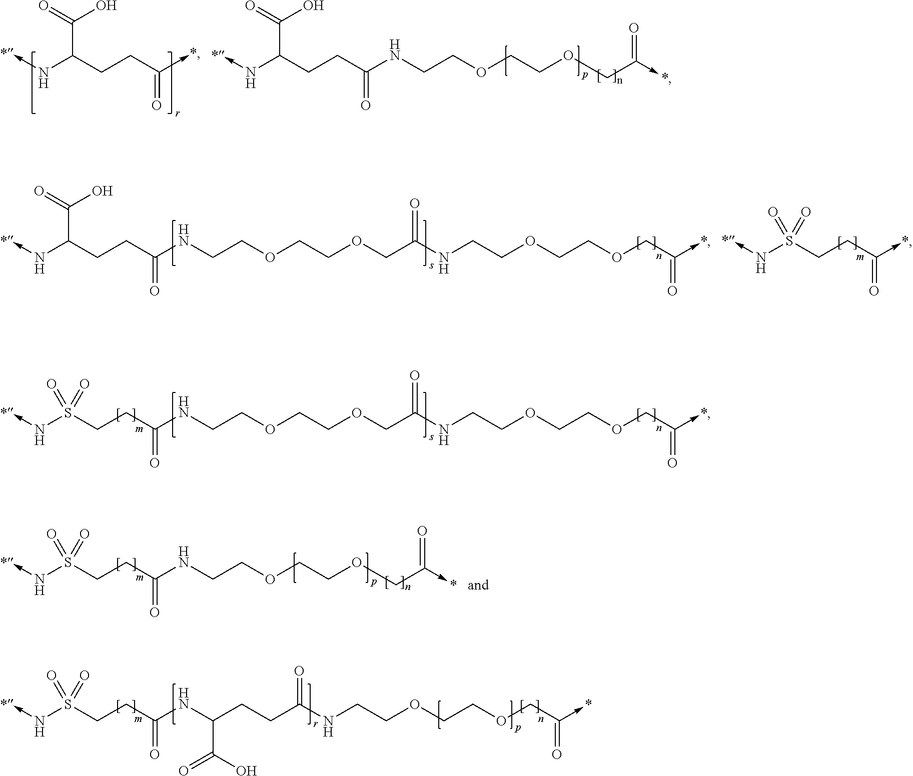

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23;
X is the attachment anchor to the Leptin compound and is

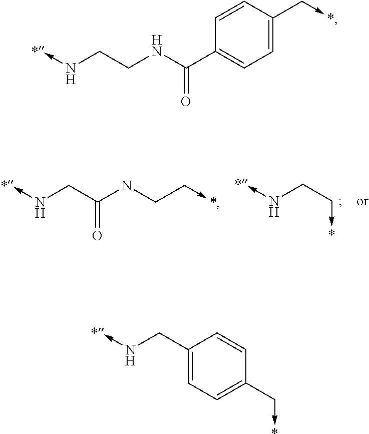

wherein “*” indicates the point of a moiety which is oriented towards the Leptin compound and “*″” indicates the point of a moiety which is oriented towards Z; or a pharmaceutical salt, amide or ester thereof.
2. A compound according to embodiment 1, wherein Y is a spacer selected from the group consisting of a bond,

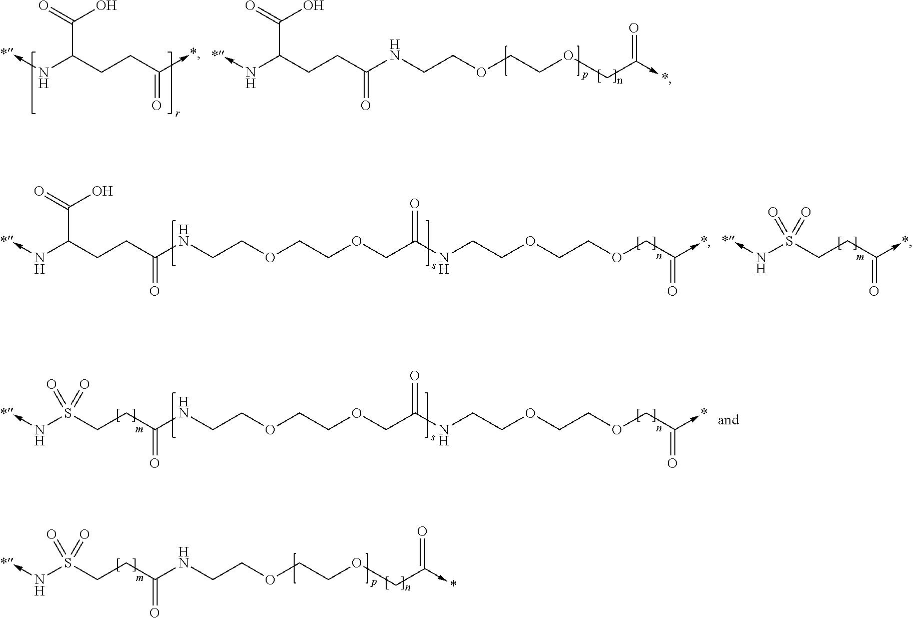

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23 and

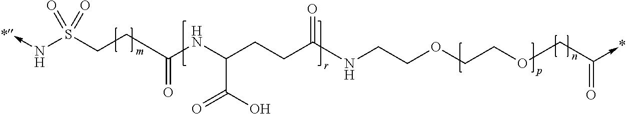

wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2 or 3.
3. A compound according to embodiment 1, wherein Y is a spacer selected from the group consisting of a bond.

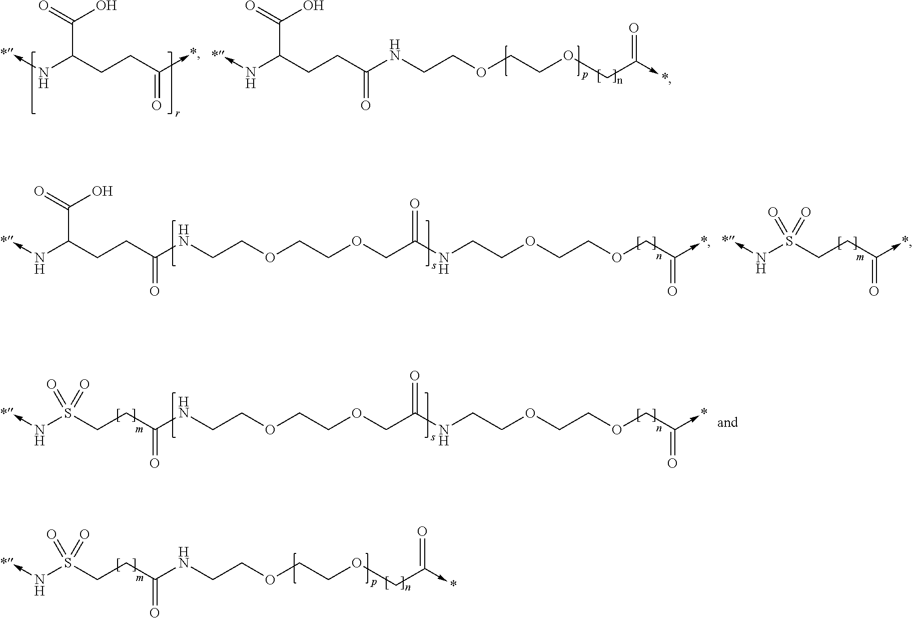

wherein m is 0, 1, 2; n is 1, 2 or 3; s is 0, 1, 2 or 3; p is 1, 2, 3 or 4; r is 1, 2 or 3 and

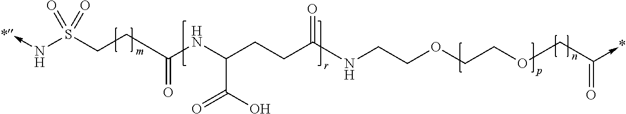

wherein m is 0, 1, 2 or 3; n is 1, 2 or 3; s is 0, 1, 2 or 3; p is 1, 2, 3 or 4 and; r is 1, 2 or 3.
4. The compound according to any one of the preceding embodiments, wherein the Z—Y—X-moiety is connected to an amino group present in an amino acid residue present in the Leptin compound or to the N terminal alpha-amino group in the Leptin compound.
5. The compound according to embodiment 1, wherein a hydrogen has been removed from said amino group.
6. The compound according to any one of the preceding embodiments, wherein said Leptin compound is an analogue of Leptin, such as an analogue of rat or human Leptin.
7. The compound according to any one of the preceding embodiments, wherein said Leptin compound has 90%, such as 95% or 98% sequence identity, to human Leptin.
8. The compound according to any one of the preceding embodiments, wherein Z comprises an acyl group.
9. The compound according to any one of the preceding embodiments, wherein Z comprises a fatty acid or fatty diacid.
10. The compound according to any one of the preceding embodiments, wherein Z comprises an alpha and omega carboxy group.
11. The compound according to any one of the preceding embodiments, wherein Z comprises a distal carboxylic acid group.
12. The compound according to any one of the preceding embodiments, wherein Z comprises a distal tetrazole group.
13. The compound according to any one of the preceding embodiments, wherein Z comprises a fatty acid or fatty diacid with 12-22 carbon atoms.
14. The compound according to any one of the preceding embodiments, wherein Z comprises a fatty acid or fatty diacid with 16-18 carbon atoms.
15. The compound according to any one of the preceding embodiments, wherein Z comprises a fatty acid or fatty diacid with 16 carbon atoms.
16. The compound according to any one of the preceding embodiments, wherein Z comprises a fatty acid or fatty diacid with 18 carbon atoms.
17. The compound according to any one of the preceding embodiments, wherein Z comprises a fatty acid or fatty diacid with 20 carbon atoms.
18. The compound according to any one of the preceding embodiments, wherein Z is

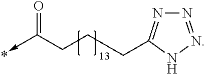

19. The compound according to any one of the preceding embodiments, wherein Y is a bond.
20. The compound according to any one of the preceding embodiments, wherein Y is

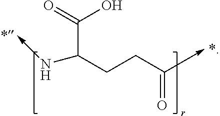

21. The compound according to any one of the preceding embodiments, wherein Y is

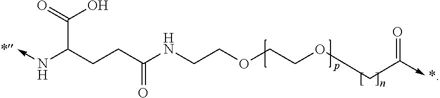

22. The compound according to any one of the preceding embodiments, wherein Y is

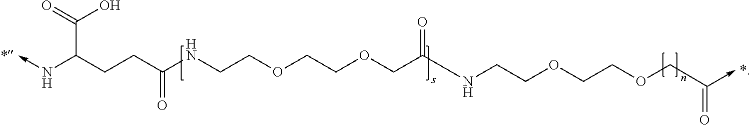

23. The compound according to any one of the preceding embodiments, wherein Y is

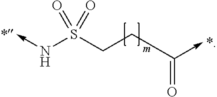

24. The compound according to any one of the preceding embodiments, wherein Y is

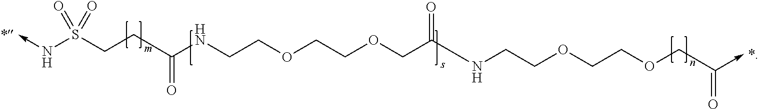

25. The compound according to any one of the preceding embodiments, wherein Y is

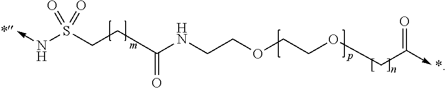

26. The compound according to any one of the preceding embodiments, wherein Y is

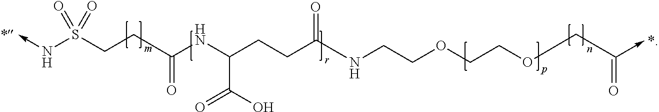

27. The compound according to any one of the preceding embodiments, wherein Y is

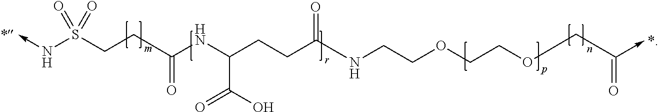

In one aspect m is 1 or 2, r is 1 or 2, p is 1, n is 1.
28. The compound according to any one of the preceding embodiments, wherein Y is

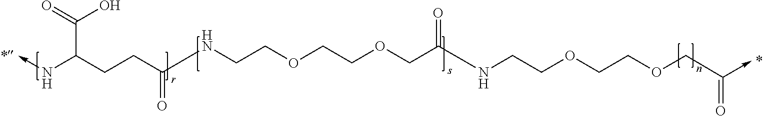

29. The compound according to any one of the preceding embodiments, wherein Y is

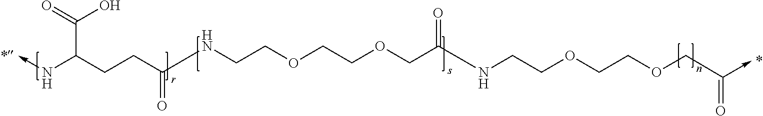

wherein r is 1, s is 1 and n is 1.
30. The compound according to any one of the preceding embodiments, wherein m is 0, 1, 2, 3, 4, 5 or 6.
31. The compound according to any one of the preceding embodiments, wherein n is 1, 2 or 3.
32. The compound according to any one of the preceding embodiments, wherein s is 0, 1, 2 or 3.
33. The compound according to any one of the preceding embodiments, wherein r is 1, 2, 3, 4, 5, 6, 7, 8, 9, 10, 11, 12, 13, 14, 15, 16, 17, 18, 19, 20, 21, 22 or 23.
34. The compound according to any one of the preceding embodiments, wherein r is 0, 1, 2 or 3.
35. The compound according to any one of the preceding embodiments, wherein p is 1, 2, 3, 4, 5, 6, 7, 8, 9, 10, 11, 12, 13, 14, 15, 16, 17, 18, 19, 20, 21, 22 or 23.
36. The compound according to any one of the preceding embodiments, wherein X is

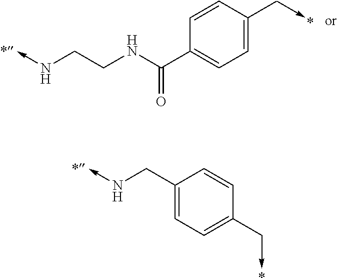

37. The compound according to any one of the preceding embodiments, wherein X is

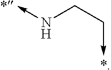

38. The compound according to any one of the preceding embodiments, wherein X is

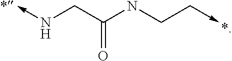

39. The compound according to any one of the preceding embodiments, wherein said compound is Compound
A:

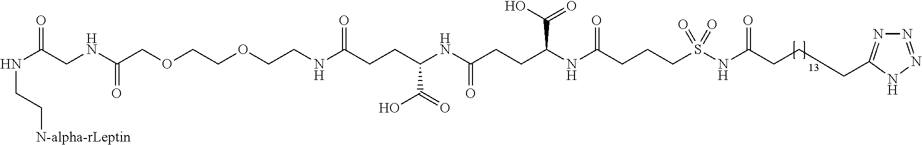

40. The compound according to any one of the preceding embodiments, wherein said compound is compound
B:

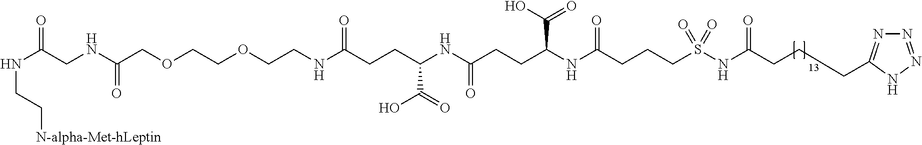

41. The compound according to any one of the preceding embodiments, wherein said compound is compound
C:

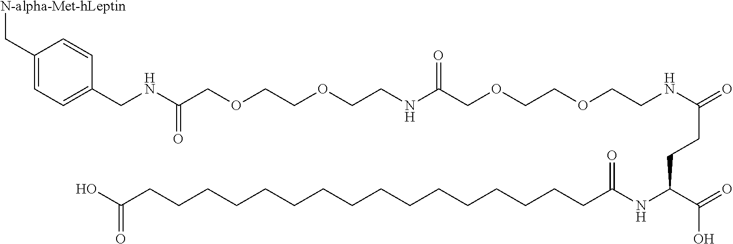

42. The compound according to any one of the preceding embodiments, wherein said compound is Compound
D:

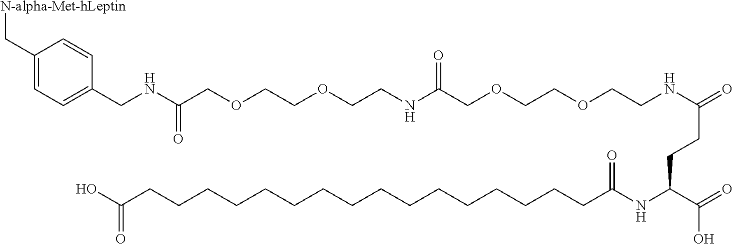

43. A compound according to any one of the preceding embodiments for use in medicine.
44. A compound according to any one of the preceding embodiments for the treatment of obesity, diabetes or lipodystrophy.
45. A composition according to embodiment 36, wherein one of said anti-obesity agents and/or anti-diabetes agents is pramlintide.
44. A compound according to any one of the preceding embodiments for the treatment of delayed puberty, amenorrhea or polycystic ovarian syndrome.
46. A compound according to any one of embodiments 41-44, wherein said compound is administered to a subject in need thereof once daily or less frequently, such as once-weekly.
47. A composition comprising a compound as defined in any one of the preceding embodiments and one or more pharmaceutical excipients.
48. Use of a compound as defined in any one of the preceding embodiments for the preparation of a medicament for the treatment of obesity, diabetes or lipodystrophy.
49. Use of a compound as defined in any one of the preceding embodiments for the preparation of a medicament for the treatment of delayed puberty, amenorrhea or polycystic ovarian syndrome.
50. A method of treatment of obesity, diabetes or lipodystrophy, wherein a compound as defined in any one of the preceding embodiments is administered to a patient in need thereof.
51. A method of treatment of delayed puberty, amenorrhea or polycystic ovarian syndrome, wherein a compound as defined in any one of the preceding embodiments is administered to a patient in need thereof.

### EXAMPLES

### Abbreviations

Boc=tert butyloxycarbonyl
CHCl₃=Chloroform
CaCl₂=Calcium Chloride
CH3CN=acetonitrile
DCM=dichloromethane, CH₂Cl₂, methylenechloride
DIC=diisopropylcarbdiimide
DIPEA=N,N-diisopropylethylamine
DMF=N,N-dimethylformamide
DMSO=dimethylsulfoxide
E. Coli=Escherichia coli
EDAC=1-ethyl-3-(3-dimethylaminopropyl)carbodiimide hydrochloride
Et₂O=diethyl ether
EtOAc=ethyl acetate
Fmoc=9H-fluoren-9-ylmethoxycarbonyl
Fmoc-Glu-O-t-Bu=N-Fmoc-glutamic acid-1-t-butyl ester
Fmoc-OEG-OH=(2[2-(Fmoc-amino)ethoxy]ethoxy)acetic acid
H₂O=water
HCl=Hydrogenchloride
HEK293=human embryonic kidney 293
HPCD=(2-Hydroxypropyl)-3-cyclodextrin
HOAt=1-hydroxy-7-azabenzotriazole
MeOH=methanol
MgCl₂=Magnesium Chloride
NaCl=sodium chloride
NMP=N-methylpyrrolidin-2-one
OEG=(2[2-(amino)ethoxy]ethoxy)acetic acid
OtBu=tert butyl ester
tBu=tert butyl
NaCl=sodium chloride
NMP=N-methylpyrrolidin-2-one
OEG=(2[2-(amino)ethoxy]ethoxy)acetic acid
OtBu=tert butyl ester
tBu=tert butyl
PBS buffer=Phosphate Buffered Saline
p-STAT-3=phosphorylated Signal transducer and activator of transcription 3
TFA=trifuloroacetic acid
TIPS=triisopropylsilane
THF=Tetrahydrofuran

### Met-hLeptin

Met-hLeptin (SEQ ID: 3SEQ ID: 3) is commercially available, however may be expressed in E. Coli. by methods known by the person skilled in the art.

### Protein Analysis Methods

UPLC: The UPLC-analysis was performed using a Waters Acquity UPLC system fitted with a Waters Acquity ACQUITY UPLC BEH C18, 1.7 um, 2.1 mm×50 mm column. UV detections were collected at 214 nm. Oven temperature was 40° C. The following eluents were used; Solvent A: 99.95% Water, 0.05% Trifluoroacetic acid

Solvent B: 99.95% Acetonitrile, 0.05% Trifluoroacetic acid. Step gradient: 5 to 35% B in 0.5 min then 35 to 55% B in 3.5 min

Gradient run-time: 4.0 minutes

Total run-time: 6.0 minutes

Flow rate: 0.45 ml/min fixed or by one of the following two methods.

TABLE 1
Model for UPLC analysis (I)

| | |
|---|---|
| System | System: Agilent 1200 series HPLC |
| | Column: Poroshell 300SB C3 2.1 × 75 mm 5 u |
| | Detector: Agilent Technologies LC/MSD TOF (G1969A) |
| Detector setup | Ionisation method: API-ES, |
| | Scanning range: m/z min. 250 m/z max. 3200 |
| | linear reflector mode |
| | positive mode |
| Conditions | Linear gradient: 25% to 95% B |
| | Gradient run-time: 20 minutes |
| | Flow rate: 0.40 ml/min fixed |
| | Column temperature: 40° C. |
| Eluents | Solvent A: 99.90% H2O, 0.1% Formic acid |
| | Solvent B: 99.90% CH3CN, 0.1% Formic acid |
| | Solvent C: NA |

TABLE 2
Model for UPLC analysis (II)

| | |
|---|---|
| System | System: Agilent 1200 series HPLC |
| | Column: Poroshell 300SB C18 2.1 × 75 mm 5 u |
| | Detector: Agilent Technologies LC/MSD TOF (G1969A) |
| Detector setup | Ionisation method: API-ES, |
| | Scanning range: m/z min. 250, m/z max. 3200 |
| | linear reflector mode |
| | positive mode |
| Conditions | Linear gradient: 5% to 95% B |
| | Gradient run-time: 20 minutes |
| | Flow rate: 0.40 ml/min fixed |
| | Column temperature: 40° C. |
| Eluents | Solvent A: 99.90% H2O, 0.1% Formic acid |
| | Solvent B: 99.90% CH3CN, 0.1% Formic acid |
| | Solvent C: NA |

### Example 1

### Synthesis of Protractor Group 4-(1-Carboxy-3-{2-[2-({[(2,2-dimethoxy-ethylcarbamoyl)-methyl]-carbamoyl}-methoxy)-ethoxy]-ethylcarbamoyl}-propylcarbamoyl)-2-[4-(16-2H-tetrazol-5-yl-hexadecanoylsulfamoyl)-butyrylamino]-butyric acid (Compound 1)

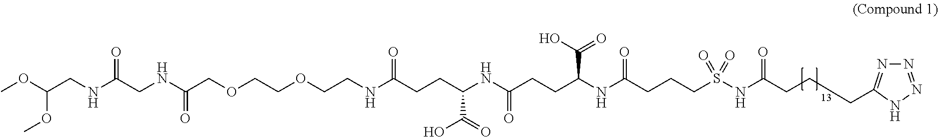

Synthesis of the protractor group compound 1 was carried as illustrated in Scheme 1 and described below.

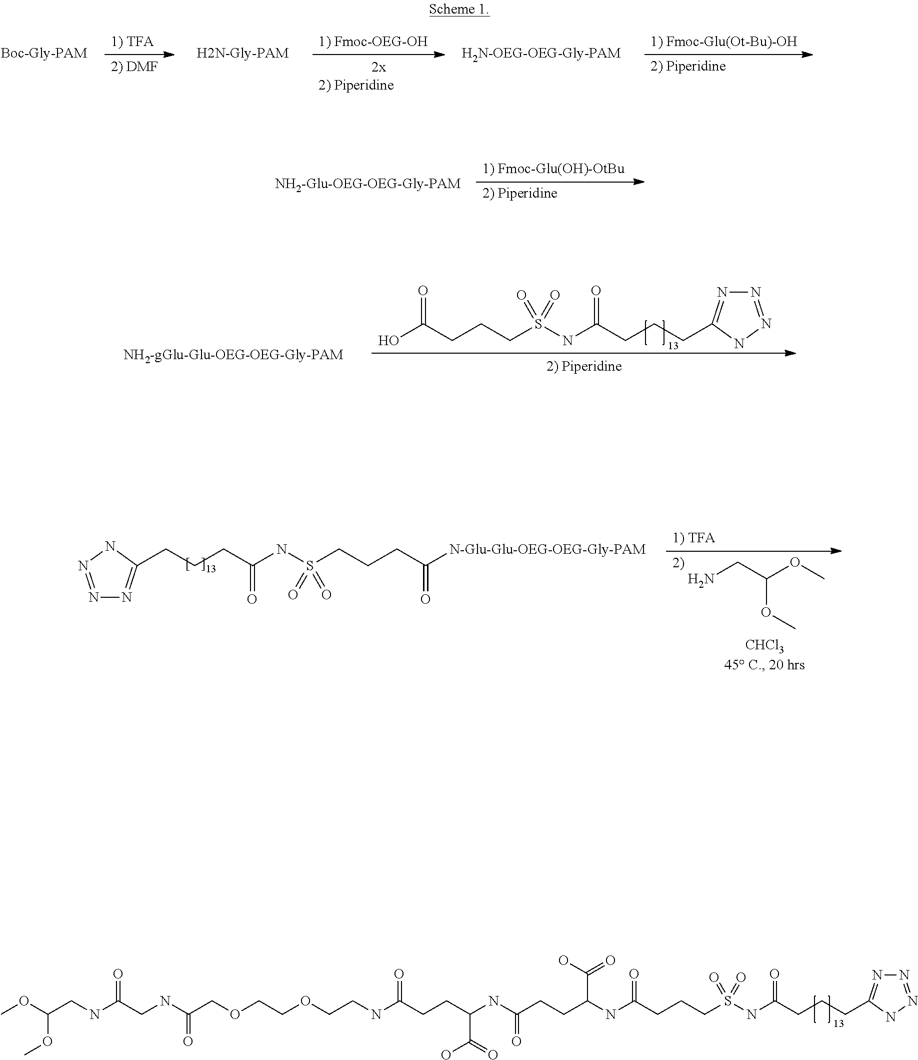

t-BOC-Gly Pam resin (3 g, loading 1 mmol/g) was washed with NMP for 1 hour and filtered. 20 mL TFA was added and the suspension was shaken for 10 minutes, filtered and washed twice with NMP, followed by wash with 5% DIPEA in NMP (25 mL) and an additional three times with NMP.

Fmoc-OEG-OH (3.1 g, 8 mmol) was dissolved in a solution of HOAt in NMP (0.25 M, 32 mL), DIC (1250 μL, 8 mmol) was added and the mixture allowed to pre-activate for 15 minutes before added to the resin. The suspension was shaken overnight, filtered and washed three times with NMP. The resin was added a solution of piperidine in NMP (25%, 20 mL) and shaken for 10 minutes, filtered and washed 6 times with NMP.

Fmoc-Glu-(OtBu)-OH (1.7 g, 4 mmol) was dissolved in a solution of HOAt in NMP (0.25 M, 16 mL), DIC (626 μL, 4 mmol) was added and the mixture allowed to pre-activate for 15 minutes before added to the resin. The suspension was shaken for 2 hours, filtered and washed three times with NMP (3 times. The resin was added a solution of piperidine in NMP (25%, 20 mL) and shaken for 10 minutes, filtered and washed 6 times with NMP.

Fmoc-Glu-(OtBu)-OH (1.7 g, 4 mmol) was dissolved in a solution of HOAt in NMP (0.25 M, 16 mL), DIC (626 μL, 4 mmol) was added and the mixture allowed to preactivate for 15 minutes before added to the resin. The suspension was shaken for 2 hours, filtered and washed three times with NMP (3 times). The resin was added a solution of piperidine in NMP (25%, 20 mL) and shaken for 10 minutes, filtered and washed 6 times with NMP.

4-(16-1H-Tetrazol-5-yl-hexadecanoylsulfamoyl)-butyric acid (synthesis described in WO2007/009894, pages 79-82) (1.9 g, 4 mmol) was dissolved in a solution of HOAt in NMP (0.25 M, 16 mL), DIC (626 μL, 4 mmol) was added and the mixture allowed to preactivate for 15 minutes before added to the resin. The suspension was shaken for 5 hours, filtered and washed three times with NMP. The resin was treated with TFA (20 mL), TIPS (500 μL) and water (500 μL) for 1 hour. The resin bound unprotected peptide was treated with a mixture of CHCl₃/aminoacetaldehydedimethylacetal (3:2) (25 mL) at 45° C. for 20 hours with a magnetic stirrer. The resin was filtered and the solution evaporated to dryness to yield the compound 1.

### Example 2

### Synthesis of Protracted Rat Leptin (Compound A)

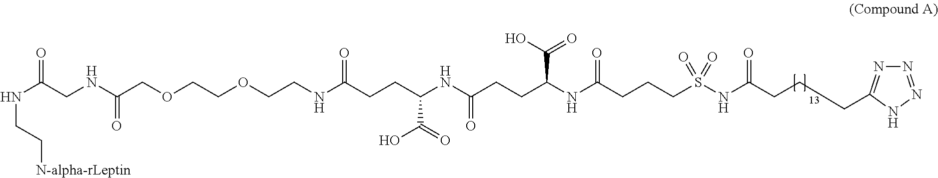

Compound 1 (22 mg, 0.0094 mmol) was dissolved in water (4 mL) containing 20% (2-Hydroxypropyl)-β-cyclodextrin (HPCD). The aldehyde was liberated by adding aq. HCl (2 μL, 1N) followed by shaking for 1 hour. Rat Leptin (100 mg, 0.0031 mmol) was dissolved in 5 mL Hepes buffer (25 mM Hepes in milliQ water, pH 7). To this solution was added the liberated aldehyde and the mixture was shaken for 1 hour at room temperature. NaCNBH₃ (50 mg) was added and the mixture was shaken overnight. The mixture was purified on a 8 mL Poros50HQ anion exchange column using a buffer consisting of 10 mM Na₂HPO₄, 15% (v/v) glycerol, pH 7.6 to which 0.5 M NaCl was added for elution conditions, which resulted in purified compound A, i.e. rat Leptin (SEQ ID NO 2) alkylated with compound 1 at the N-terminal amino group. Mw=17190 g/mol.

UPLC and LC-MS analysis was carried out as described above and the results are UPLC: RT=3.66 min; LC-MS: Average mass=17190.2 Da (calculated=17189.8 Da; MS Resolution=100000).

### Example 3

### Synthesis of Protracted Human Leptin (Compound B)

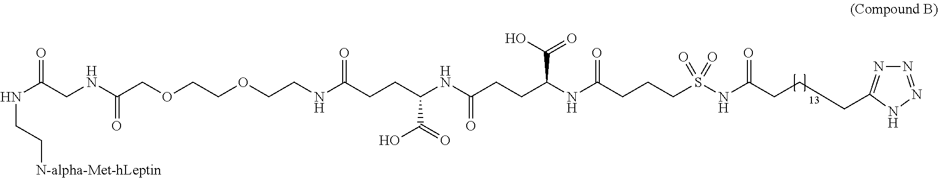

Compound 1 (10 mg) was dissolved in water (2 mL) containing 20% (2-Hydroxypropyl)-β-cyclodextrin (HPCD). The aldehyde was liberated by adding aq. HCl (1 μL, 1N) followed by shaking for 1 hour. Met-hLeptin (50 mg) was dissolved in a mixture of 2.5 mL Hepes buffer (25 mM Hepes in milliQ water, pH 7)+1.5 mL Hepes buffer (25 mM, pH 7.0) containing 5% HPCD). To this solution was added the liberated aldehyde and the mixture was shaken for 24 hour at room temperature. NaCNBH₃ (24 mg) was added and the mixture was shaken overnight. The mixture was purified on a 8 mL Poros50HQ anion exchange column using a buffer consisting of 10 mM Na₂HPO₄, 15% (v/v) glycerol, pH 7.6 to which 0.5 M NaCl was added for elution conditions, which resulted in purified compound B, i.e. human Leptin (SEQ ID NO 3) alkylated with compound 1 at the N-terminal amino group. Mw=17115 g/mol.

UPLC and LC-MS analysis was carried out as described above and the results are UPLC: RT=3.72 min; LC-MS: Average mass=17115 Da.

### Example 4

### Synthesis of Protractor Group 17-[(S)-1-Carboxy-3-(2-{2-[(2-{2-[(4-formyl-benzylcarbamoyl)-methoxy]-ethoxy}-ethylcarbamoyl)-methoxy]-ethoxy}ethylcarbamoyl)-propylcarbamoyl]-heptadecanoic acid (Compound 2)

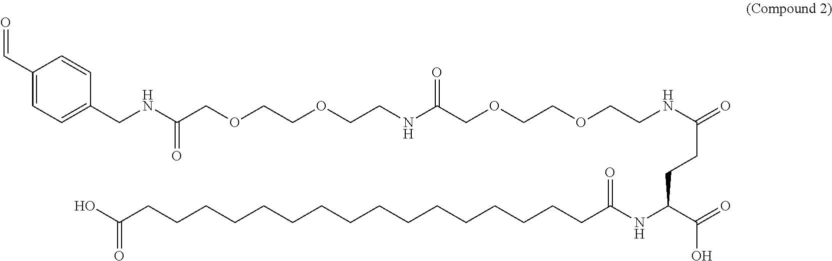 [SMILES: O=Cc1ccc(CNC(=O)COCCOCCNC(=O)COCCOCCNC(=O)CC[C@H](NC(=O)CCCCCCCCCCCCCCCCC(=O)O)C(=O)O)cc1]

Synthesis of the protractor group compound 2 was carried as illustrated in Scheme 2 and described below.

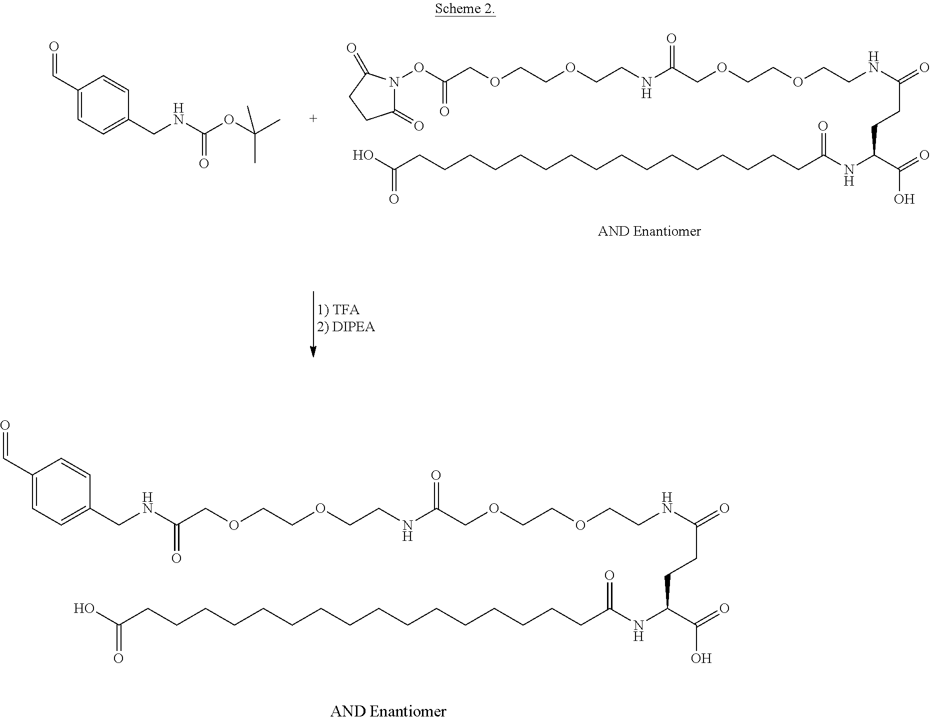 [SMILES: CC(C)(C)OC(=O)NCc1ccc(C=O)cc1 ; O=C(O)CCCCCCCCCCCCCCCCC(=O)N[C@@H](CCC(=O)NCCOCCOCC(=O)NCCOCCOCC(=O)ON1C(=O)CCC1=O)C(=O)O ; O=Cc1ccc(CNC(=O)COCCOCCNC(=O)COCCOCCNC(=O)CC[C@H](NC(=O)CCCCCCCCCCCCCCCCC(=O)O)C(=O)O)cc1]

t-Bu-N-(4-formyl-benzyl) carbamate (100 mg) was treated with TFA/DCM (1:1) for 1 h. The mixture was concentrated in vacuo and co-concentrated with toluene (twice).

The residue was dissolved in THF (2.5 ml) and a solution of 17-((S)-1-Carboxy-3-{2-[2-({2-[2-(2,5-dioxo-pyrrolidin-1-yloxycarbonylmethoxy)-ethoxy]-ethylcarbamoyl}-methoxy)-ethoxy]-ethylcarbamoyl}-propylcarbamoyl)-heptadecanoic acid (320 mg) in THF (5 ml) was added. DIPEA (0.5 ml) was added slowly. After 130 min, the mixture was concentrated in vacuo.

The residue was dissolved in EtOAc and 1N HCl. The organic layer was extracted with 1N HCl and brine. The organic layer was dried (Na2SO4) and concentrated in vacuo to give a white solid.

Synthesis of 17-((S)-1-Carboxy-3-{2-[2-({2-[2-(2,5-dioxo-pyrrolidin-1-yloxycarbonylmethoxy)-ethoxy]-ethylcarbamoyl}-methoxy)-ethoxy]-ethylcarbamoyl}-propylcarbamoyl)-heptadecanoic was performed according to the method described in WO2009083549 example 7, page 79.

### Example 5

### Synthesis of Protracted Rat Leptin (Compound C)

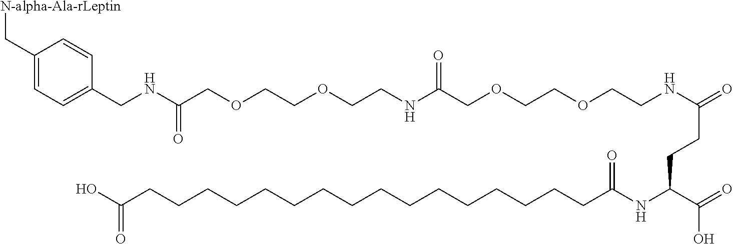

General Procedure:

The Leptin was transferred to a phosphate buffer, pH ˜7.4, concentration 5-10 mg/mL.

The protractor (i.e. Compound 2) was dissolved in a 40% HPβCD solution at a concentration of 10 mg/mL. 4 equivalents of the protractor was added to the protein. Total volume ˜5 mL.

A fresh solution of NaCNBH₃ in methanol was prepared (5-10%). Several aliquots of 50 μL of the reducing agent in methanol was added during the next two days to the protein solution (˜200 μL per 24 h). The reaction was monitored using an LC-MS. On the third day, the product was purified using a HIC column and a gradient of 10× PBS vs. MilliQ water. The purified product was compound C, i.e. rat Leptin (SEQ ID NO 2) alkylated with compound 2 at the N-terminal amino group.

Protracted rLeptin

LC-MS: calc mass 17065.72, found 17068.33

### Example 6

### Synthesis of Protracted Human Leptin (Compound D)

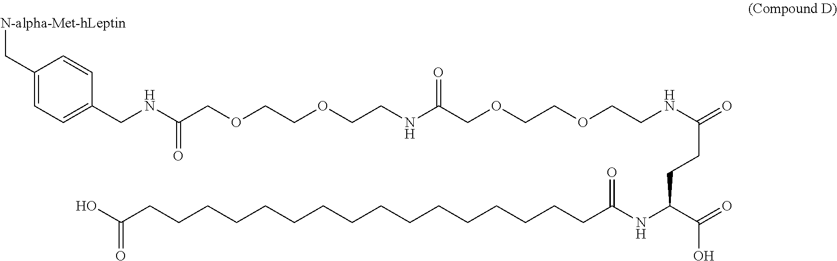

The Met-hLeptin (SEQ ID NO: 3) was transferred to a phosphate buffer, pH ˜7.4, concentration 5-10 mg/mL.

The protracto (i.e. Compound 2) was dissolved in a 40% HPβCD solution at a concentration of 10 mg/mL. 4 equivalents of the protractor was added to the protein. Total volume ˜5 mL.

A fresh solution of NaCNBH₃ in methanol was prepared (5-10%). Several aliquots of 50 μL of the reducing agent in methanol was added during the next two days to the protein solution (˜200 μL per 24 h). The reaction was monitored using an LC-MS. On the third day, the product was purified using a HIC column and a gradient of 10× PBS vs. MilliQ water.

The purified product was compound D, i.e. Met-hLeptin (SEQ ID NO: 3) alkylated with compound 2 at the N-terminal amino group.

### Protracted Met-hLeptin

LC-MS: calc mass 16990.60, found 16992.44

### Example 7

### Whole Cell Binding of Compounds According to Examples 5 and 6 (Compound C and D)

HEK293 cells stably expressing the human Leptin receptor were seeded in poly-D-lysine coated 24 well plates at 200.000 cells per well and cultured for two days in alpha-minimum essential medium (MEM), cell culture media containing 10% heat inactivated fetal calf serum (FCS), 1% penicillin-streptomycin (P/S), 1 mg/ml Zeocin and 1 mg/ml G418 antibiotic at +37° C. in a humidified atmosphere with 5% CO₂. Prior to the experiment, cells were rinsed in pure MEM medium, followed by incubation in Leptin analogues at 10,3,1,0.3,0.1,0.03 and 0.01 nM concentrations for 45 minutes in MEM containing 0.005% polysorbate 20 and 0.1% ovalbumin and [¹²⁵I]-hLeptin 100000 cpm. The cells were washed three times in ice cold MEM and were lysed in lysis buffer containing 1.0% nonidet P-40, 0.5% triton X-100 (C₁₄H₂₂O(C₂H₄O)ₙ) and 1M sodium hydroxide. Samples are transferred to plastic

TABLE 3
Whole cell binding of compound according to the present invention.
Example 8: Scintillation Proximity Assay (SPA) of compounds according
to examples 2, 3, 5 and 6 (Compound A, B, C and D)

| Whole cell binding | Mean IC50 nM | n= | SEM |
|---|---|---|---|
| Human Leptin | 0.25 | 4 | 0.02 |
| Compound D | 0.61 | 3 | 0.03 |
| Compound B | N/A | | |
| Rat Leptin | 1.01 | 4 | 0.19 |
| Compound C | N/A | | |
| Compound A | 1.76 | 4 | 0.10 |

HEK 293 cells stably expressing the human Leptin receptor were cultured in 500 cm² cell harvesting dishes in RPMI 1640 cell culture media containing 10% heat inactivated fetal calf serum, 1% penicillin-streptomycin (P/S), 1 mg/ml Zeocin and 1 mg/ml G418 antibiotic at +37° C. in a humidified atmosphere with 5% CO₂ and detached mechanically by scraping. Plates were washed in ice cold PBS (137 mM NaCl, 2.7 mM KCl, 4.3 mM Na₂HPO₄, 1.47 mM KH₂PO₄ pH adjusted to 7.4) and cells were transferred to tubes and centrifuged for 5 min at 1000 g at +4° C. Pellets were re-suspended in ice cold homogenization buffer (20 mM Hepes, 5 mM MgCl₂, 1 mg/ml Bacitracin, pH 7.1) and then homogenized for 30 seconds using a tissue homogenizer at medium speed. The homogenate was centrifuged at 35000 g using an ultracentrifuge for 10 minutes at +4° C. and the supernatant was discarded and fresh homogenization buffer added. Homogenization of the pellet was repeated a total of three times. The final pellet was re-suspended in a few millilitres of homogenization buffer and protein concentration was determined using the Bradford method and measured at 595 nm on a microplate reader. Protein concentration were adjusted to 1 mg/ml and transferred to cryotubes and stored at −80° C.

Human Leptin receptor SPA binding assay were performed in white 96-well plates in a total volume of 200 μl per well. Wheat germ agglutinin coated beads containing scintillation liquid were reconstituted in binding buffer (50 mM Hepes, 1 mM CaCl₂, 5 mM MgCl₂, 0.02% Tween 20, 0.25% Ovalbumin pH 7.4) and mixed with membrane preparation to give final concentration of 1 mg beads and 10 μg total protein per well. 50.000 cpm per well of radio ligand human [¹²⁵I]-Leptin was added corresponding to a concentration of approximately 100 μM. Human serum albumin was added to a final concentration of 2% when binding in presence of albumin was investigated. Freeze dried Leptin analogues were dissolved in PBS to 100 μM and serial diluted in binding buffer to give a final assay concentration ranging from 100 nM to 0.01 μM. The plate was sealed and incubated at +25° C. for 2 hours in a plate shaker set at 400 rpm and thereafter centrifuged at 1500 rpm for 10 minutes prior to reading of luminescence on a microplate scintillation and luminescence counter. Displacement of radioligand was measured as reduction in luminescence and IC₅₀ values were calculated by nonlinear regression analysis of sigmoidal dose-response curves.

TABLE 4
SPA binding of compounds according to the present invention.

| 0% HSA added | | | | 2% HSA added | | |
|---|---|---|---|---|---|---|
| SPA binding | Mean IC50 nM | n= | SEM | Mean IC50 nM | n= | SEM |
| Human Leptin | 0.21 | 3 | 0.02 | 0.40 | 3 | 0.06 |
| Compound D | 0.23 | 3 | 0.04 | 5.38 | 3 | 1.40 |
| Compound B | 1.26 | 3 | 0.22 | 3.40 | 3 | 0.90 |
| Rat Leptin | 0.49 | 3 | 0.04 | 0.88 | 3 | 0.18 |
| Compound C | 0.38 | 3 | 0.02 | 5.93 | 3 | 1.80 |
| Compound A | 1.90 | 3 | 0.51 | 37.12 | 3 | 16.08 |

### Example 9

### Functional Luciferase Assay of Compounds According to Examples 2, 3, 5 and 6 (Compound A, B, C and D)

HEK293 cells stably expressing the hLeptin receptor and p-STAT-3 response element with a Luciferase reporter gene were cultured in RPMI 1640 cell culture media containing 10% heat inactivated fetal calf serum (FCS), 1% penicillin-streptomycin (P/S), 1 mg/ml Zeocin and 1 mg/ml G418 antibiotic at +37° C. in a humidified atmosphere with 5% CO₂. Cells were seeded in a 96 well plate (20.000 cells per well) and let to attach for 24 hours followed by starvation in RPMI medium with 1% penicillin-streptomycin (P/S) only for 24 hours. Cells were incubated in Leptin analogues without or with 0,7% human serum albumin at final concentration ranging from 100 nM to 0.01 μM in RPMI medium with 1% penicillin-streptomycin (P/S) for 4.5 hours followed by removal of all medium. Luciferase catalyzes the oxidation of the firefly-specific substrate, D-luciferin, to produce light and a lysis buffer containing D-luciferin were diluted 1:1 with PBS and 200 μl was added to each well followed by 30 minutes incubation in room temperature. Luminescence was measured on a microplate scintillation and luminescence counter and EC₅₀ values were calculated by nonlinear regression analysis of sigmoidal dose-response curves.

TABLE 5
Luciferase assay of compound according to the present invention.

| 0% HSA added | | | | | | |
|---|---|---|---|---|---|---|
| | Mean EC50 | | | 2% HSA added | | |
| Luciferase assay | nM | n= | SEM | Mean IC50 nM | n= | SEM |
| Human Leptin | 0.23 | 8 | 0.11 | 0.18 | 4 | 0.14 |
| Compound D | 0.29 | 8 | 0.06 | 0.45 | 4 | 0.05 |
| Compound B | 0.61 | 4 | 0.12 | 0.27 | 2 | 0.04 |
| Rat Leptin | 0.41 | 7 | 0.19 | 0.52 | 4 | 0.43 |
| Compound C | 0.24 | 4 | 0.01 | 0.42 | 3 | 0.04 |
| Compound A | 1.57 | 7 | 0.37 | 1.92 | 4 | 0.29 |

### Example 10

### Humas Serum Albumin (HSA) Binding of Compounds According to Examples 2, 3, 5 and 6 (Compound A, B, C and D) at HSA Concentrations 0.7% and 2.0%

TABLE 6
HAS binding of compounds according to the present invention.

| 2% HSA | | | 0.7% HSA | | |
|---|---|---|---|---|---|
| Mean IC50 nM | n= | SEM | Mean IC50 nM | n= | SEM |
| 0.40 | 3 | 0.06 | 0.18 | 4 | 0.14 |
| 5.38 | 3 | 1.40 | 0.45 | 4 | 0.05 |
| 3.40 | 3 | 0.90 | 0.27 | 2 | 0.04 |
| 0.88 | 3 | 0.18 | 0.52 | 4 | 0.43 |
| 5.93 | 3 | 1.80 | 0.42 | 3 | 0.04 |
| 37.12 | 3 | 16.08 | 1.92 | 4 | 0.29 |

### Pharmacological Methods

Assay (I): Experimental Procedure for Monitoring Food Intake and Body Weight in ob/ob Mice

Food intake was monitored in ob/ob mice housed individually after single dose of Leptin. Continuous food intake was monitored automatically via an online food intake monitoring system (BioDAQ). The system contained 32 places with individual food hoppers placed on sensitive scales. Whenever food was removed from the food hopper this was recorded by the computer which continuously collected data from each of the 32 individual scales.

The mice were 8-9 months old ob/ob mice (Taconic) when administered sub cutaneously (s.c) with Leptin. They had been acclimatised to the system for more than two weeks before onset of the experiment. They were housed undisturbed in reversed day-night light cycle (dark from 10 am to 10 pm). There were two mice per cage, these were separated with a dividing wall allowing for some interaction between two mice while at the same time making it possible to make individual food intake recordings.

The mice were fed chow (D12450B from Research Diets). The pellets were placed in food hoppers made for the scales and allowing the mice to eat ad libitum without wasting excess food outside the scales. The mice had free access to water.

The mice were fasted for 4 h and dosed once s.c. 30 min before onset of dark with a composition comprising Leptin.

Food intake was monitored for a period after dosing. The body weight was obtained prior to dosing and at a time point thereafter, such as at day six. Differences in food intake and body weight were statistically evaluated by one-way ANOVA analysis, followed by Dunetts post test to compare to vehicle treatment.

### Assay (II):

Food intake and body weight of ob/ob mice was measured over 6 days according to Assay (I) after administration of vehicle, wt rat/human Leptin or Compounds A/B/C/D. The dose volume was 0.2 or 0.6 ml per mouse. The results are shown in Table 2 (food intake) and Table 3 (body weight). It was observed that the Leptin derivative according to the invention had significant and long lasting effect on food intake in ob/ob mice. It was observed that the Leptin derivative according to the invention had significant and dose dependent effect on reduction of body weight in ob/ob mice. Furthermore, it was observed that the natural diurnal rhythm was intact.

### Assay (III):

Blood samples (10 ul) were taken in capillary tubes from the tail vein at various time points (see table). The capillary tubes were placed into tubes containing 500 ul EBIO (EBIO Eppendorf, Germany) buffer and blood glucose concentration was analysed in the BioSen (EKF Diagnostics). The blood glucose concentration was determined by a glucose analyzer (Biosen 5030, EKF Diagnostic, Germany).

### Example 11

### Food Intake in ob/ob Mice After Administration of 60 or 180 ug/Mice Compounds According to Examples 2 (Compound A)

TABLE 7
Effect on food intake One-way-ANOVA analysis was performed with
Dunnetts multiple comparison test, where * represents p < 0.05, **
represents p < 0.01 and *** represents p < 0.001 relative to vehicle.
Food intake (g)
mean ± SEM

| | | SEQ ID NO: 2 | Compound A | Compound A |
|---|---|---|---|---|
| | Vehicle | 144 ug/mice | 60 ug/mice | 180 ug/mice |
| | n = 8 | n = 8 | n = 7 | n = 8 |
| 0-24 h | 3.3 ± 0.15 | 2.4 ± 0.19 *** | 2.4 ± 0.13 *** | 2.4 ± 0.08 *** |
| 24-48 h | 3.1 ± 0.20 | 3.0 ± 0.12 | 1.4 ± 0.17 *** | 1.4 ± 0.19 *** |
| 48-72 h | 3.4 ± 0.14 | 3.3 ± 0.12 | 1.0 ± 0.24 *** | 1.1 ± 0.22 *** |
| 72-96 h | 3.9 ± 0.22 | 3.6 ± 0.35 | 2.0 ± 0.24 ** | 1.4 ± 0.39 *** |
| 96-120 h | 3.5 ± 0.07 | 3.5 ± 0.11 | 3.3 ± 0.16 | 2.8 ± 0.41 |
| 120-135 h | 3.0 ± 0.19 | 2.8 ± 0.12 | 3.1 ± 0.10 | 3.2 ± 0.19 |

### Example 12

### Body Weight Change of in ob/ob Mice After Administration of 60 or 180 ug/Mice Compounds According to Examples 2 (Compound A)

TABLE 8
Effect on body weight One-way-ANOVA analysis was performed with Dunnetts
multiple comparison test, where * represents p < 0.05, ** represents p < 0.01
and *** represents p < 0.001 relative to vehicle.
Body Weight change (g)
mean ± SEM

| | | SEQ ID NO: 2 | Compound A | Compound A |
|---|---|---|---|---|
| | Vehicle | 144 ug/mice | 60 ug/mice | 180 ug/mice |
| | n = 8 | n = 8 | n = 7 | n = 8 |
| Body Weight | −0.1 ± 0.17 | −0.5 ± 0.17 | −3.4 ± 0.41 *** | −4.4 ± 0.4 *** |
| change day 6 | | | | |

### Example 13

### Food Intake in ob/ob Mice After Administration of 150 ug/Mice Compounds According to Examples 2 and 3 (Compound A and B)

TABLE 9
One-way ANOVA was performed with Bonferoni's Multiple comparison test, where */†
represents p < 0.05, **/†† represents p < 0.01 and ***/††† represents p < 0.001 relative
to vehicle. * represents the significance of vehicle vs. drug, † represents the significance
of protracted rat/human Leptin vs. rat/human native Leptin
Food intake (g)
mean ± SEM

| | | (SEQ ID NO: 2) | Compound A | (SEQ ID NO: 3) | Compound B |
|---|---|---|---|---|---|
| | Vehicle | n = 6 | n = 7 | n = 6 | n = 6 |
| Time | n = 6 | 150 μg/mouse | 150 μg/mouse | 150 μg/mouse | 150 μg/mouse |
| 0-24 h | 4.06 ± 0.29 | 3.37 ± 0.34 | 2.71 ± 0.21** | 3.25 ± 0.19 | 2.57 ± 0.25** |
| 24-48 h | 4.07 ± 0.29 | 4.57 ± 0.45 | 1.42 ± 0.13*/††† | 3.94 ± 0.12 | 1.97 ± 0.58**/†† |
| 48-72 h | 4.24 ± 0.33 | 4.68 ± 0.38 | 1.03 ± 0.17***/††† | 4.04 ± 0.27 | 1.74 ± 0.51***/††† |
| 72-96 h | 4.21 ± 0.20 | 4.74 ± 0.27 | 1.67 ± 0.28***/††† | 4.00 ± 0.26 | 2.84 ± 0.20**/† |
| 96-120 h | 4.16 ± 0.14 | 5.04 ± 0.44 | 3.26 ± 0.33†† | 4.63 ± 0.32 | 4.00 ± 0.16 |
| 120-144 h | 4.17 ± 0.18 | 5.50 ± 0.68 | 4.04 ± 0.29 | 5.08 ± 0.53 | 4.26 ± 0.28 |

### Example 14

### Body Weight Change in ob/ob Mice After Administration of 150 ug/Mice Compounds According to Examples 2 and 3 (Compound A and B)

TABLE 10
One-way ANOVA was performed with Bonferoni's Multiple comparison test, where */†
represents p < 0.05, **/†† represents p < 0.01 and ***/††† represents p < 0.001 relative
to vehicle. * represents the significance of vehicle vs. drug, † represents the significance
of protracted rat/human Leptin vs. rat/human native Leptin
Body Weight change (g) and blood glucose change (mmol/l)
mean ± SEM

| | | (SEQ ID NO: 2) | Compound A | (SEQ ID NO: 3) | Compound B |
|---|---|---|---|---|---|
| | Vehicle | n = 6 | n = 7 | n = 6 | n = 6 |
| | n = 6 | 150 μg/mouse | 150 μg/mouse | 150 μg/mouse | 150 μg/mouse |
| Body Weight | −0.90 ± 0.27 | −0.81 ± 0.70 | −5.72 ± 0.35***/††† | −0.99 ± 0.39 | −4.08 ± 0.40***/††† |
| change day 6 | | | | | |
| Blood glucose | −2.42 ± 0.69 | 0.49 ± 1.16 | −5.73 ± 0.97††† | 1.57 ± 1.07* | −4.47 ± 0.63††† |
| change day 6 | | | | | |

### Example 15

### Food Intake in ob/ob Mice After Administration of 150 ug/Mice Compounds According to Examples 4 and 5 (Compound C and D)

TABLE 11
One-way ANOVA was performed with Bonferoni's Multiple comparison test, where */†
represents p < 0.05, **/†† represents p < 0.01 and ***/††† represents p < 0.001 relative
to vehicle. * represents the significance of vehicle vs. drug, † represents the significance
of protracted rat/human Leptin vs. rat/human native Leptin
Food intake (g)
mean ± SEM

| | | (SEQ ID NO: 2) | Compound C | (SEQ ID NO 3) | Compound D |
|---|---|---|---|---|---|
| | Vehicle | n = 6 | n = 6 | n = 6 | n = 6 |
| Time | n = 5 | 150 μg/mouse | 150 μg/mouse | 150 μg/mouse | 150 μg/mouse |
| 0-24 h | 3.96 ± 0.13 | 2.95 ± 0.29 | 2.79 ± 0.24 | 3.31 ± 0.61 | 3.05 ± 0.30 |
| 24-48 h | 3.54 ± 0.33 | 2.94 ± 0.22 | 0.68 ± 0.15***/††† | 3.27 ± 0.37 | 0.84 ± 0.21***/††† |
| 48-72 h | 3.24 ± 0.33 | 3.49 ± 0.28 | 0.52 ± 0.26***/††† | 3.24 ± 0.40 | 1.18 ± 0.26***/††† |
| 72-96 h | 3.59 ± 0.24 | 4.20 ± 0.32 | 2.72 ± 0.27 † | 4.83 ± 0.46 | 2.85 ± 0.30ns †† |
| 96-120 h | 3.63 ± 0.34 | 4.10 ± 0.20 | 3.12 ± 0.13 | 4.72 ± 0.67 | 3.83 ± 0.31 |
| 120-144 h | 3.69 ± 0.31 | 3.80 ± 0.29 | 3.42 ± 0.30 | 3.96 ± 0.20 | 3.36 ± 0.23 |
| 144-168 h | 3.44 ± 0.24 | 3.63 ± 0.29 | 3.66 ± 0.20 | 3.91 ± 0.50 | 3.63 ± 0.19 |

### Example 15

### Food Intake in ob/ob Mice After Administration of 150 ug/Mice Compounds According to Examples 4 and 5 (Compound C and D)

TABLE 12
One-way ANOVA was performed with Bonferoni's Multiple comparison test, where */†
represents p < 0.05, **/†† represents p < 0.01 and ***/††† represents p < 0.001 relative
to vehicle. * represents the significance of vehicle vs. drug, † represents the significance
of protracted rat/human Leptin vs. rat/human native Leptin
Body Weight change (g) and blood glucose change (mmol/l)
mean ± SEM

| | | (SEQ ID NO 2) | Compound C | (SEQ NO 3) | Compound D |
|---|---|---|---|---|---|
| | Vehicle | n = 6 | n = 7 | n = 6 | n = 6 |
| | n = 6 | 150 μg/mouse | 150 μg/mouse | 150 μg/mouse | 150 μg/mouse |
| Body Weight | −0.15 ± 0.93 | −0.69 ± 0.36 | −2.82 ± 0.44* | −0.71 ± 0.35 | −2.57 ± 0.549 * |
| change day 6 | | | | | |
| Blood glucose | 2.32 ± 1.29 | −0.41 ± 1.14 | −5.32 ± 1.32***/† | −1.60 ± 1.01 | −2.82 ± 0.99 * |
| change day 6 | | | | | |

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1. A compound or a pharmaceutical salt, amide or ester thereof comprising a Z—Y—X-moiety and a Leptin compound having the general formula Z—Y—X-Leptin compound wherein
Z is an acyl group comprising 12-22 carbon atoms and a C-terminal carboxylic acid or a C-terminal tetrazole group;
Y is a spacer selected from the group consisting of a bond, 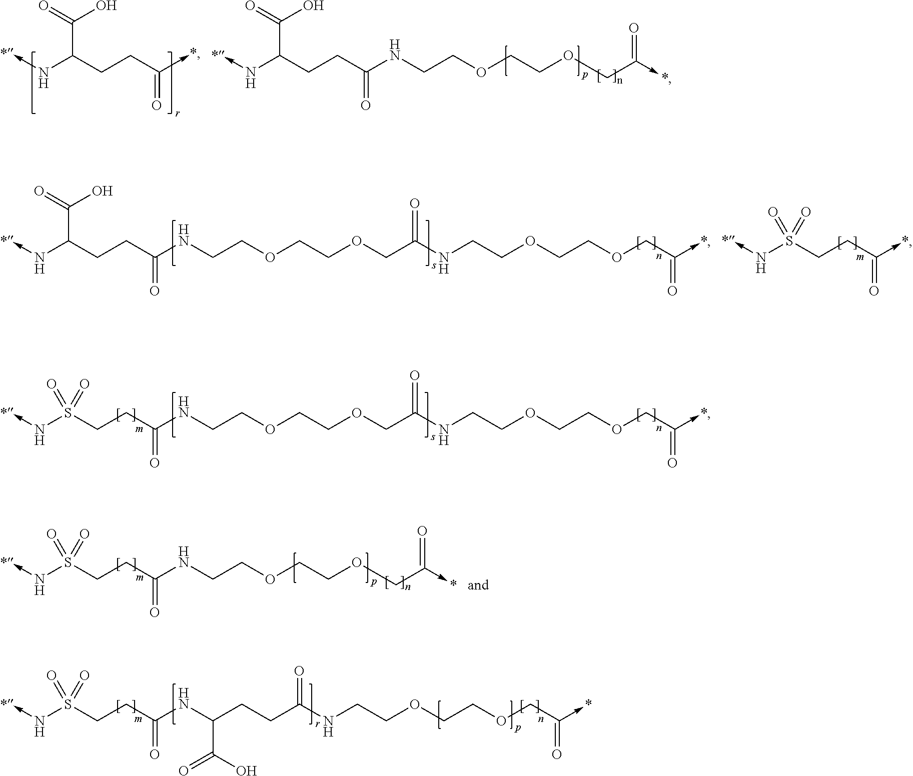
wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23;
X is the attachment anchor to the Leptin compound and is selected from the group consisting of 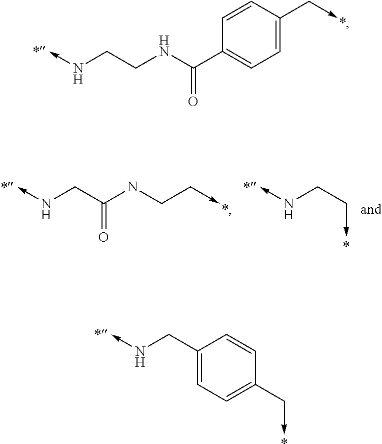
wherein “*” indicates the point of a moiety which is oriented towards the leptin compound and “*″” indicates the point of a moiety which is oriented towards Z.

2. A compound according to claim 1, wherein the Z—Y—X-moiety is connected to an amino group present in the N terminal alpha-amino group of the Leptin compound.

3. A compound of the general formula Z—Y—X-Leptin compound, wherein
Z is an acyl group comprising 12-22 carbon atoms and a
C-terminal carboxylic acid or a C-terminal tetrazole group;
Y is a spacer selected from the group consisting of a bond, 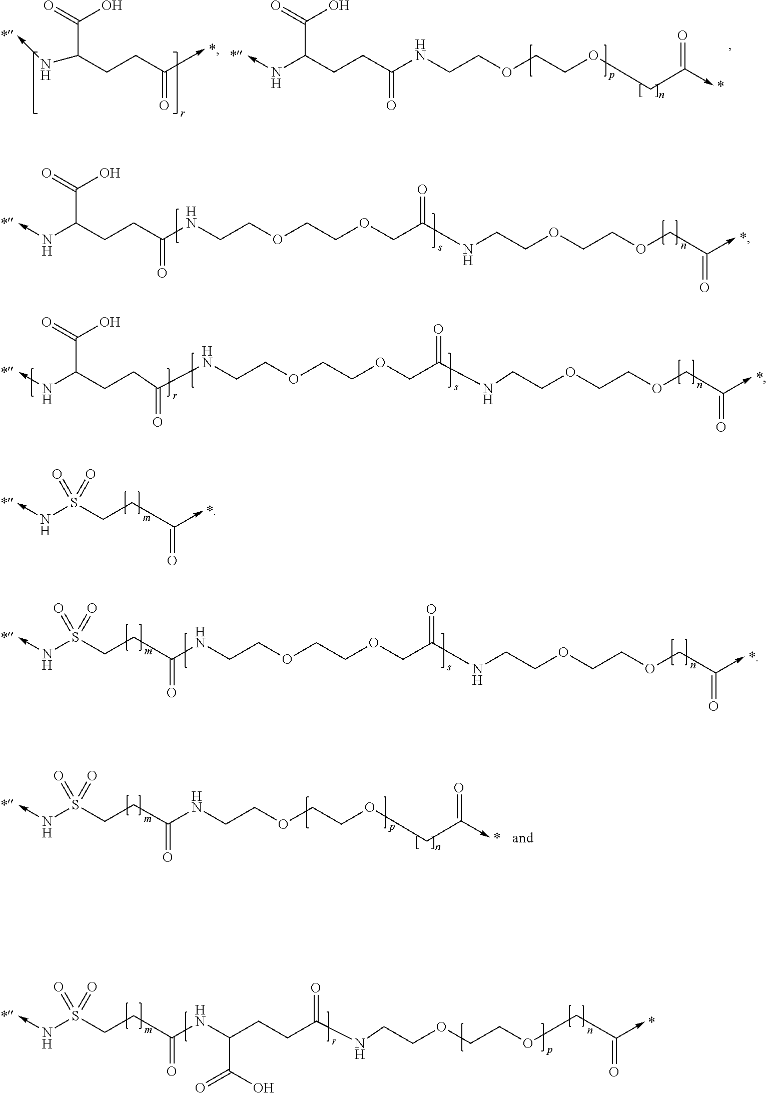
wherein m is 0, 1, 2, 3, 4, 5 or 6; n is 1, 2 or 3; s is 0, 1, 2 or 3; p is 1, 2, 3, 4, 5, 6, 7, 8, 9, 10, 11, 12, 13, 14, 15, 16, 17, 18, 19, 20, 21, 22 or 23; r is 1, 2, 3, 4, 5, 6, 7, 8, 9, 10, 11, 12, 13, 14, 15, 16, 17, 18, 19, 20, 21, 22 or 23; and
X is the attachment anchor to the Leptin compound and is selected from the group consisting of:

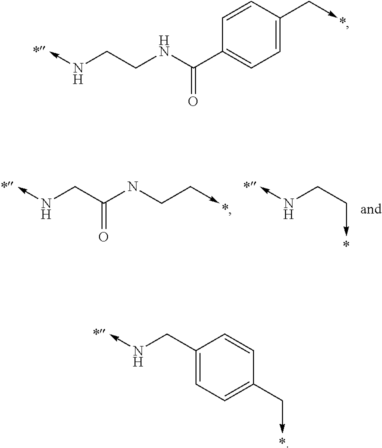

4. A compound according to claim 1,
wherein Z comprises 16-18 carbon atoms;
Y is a spacer selected from the group consisting of a bond, 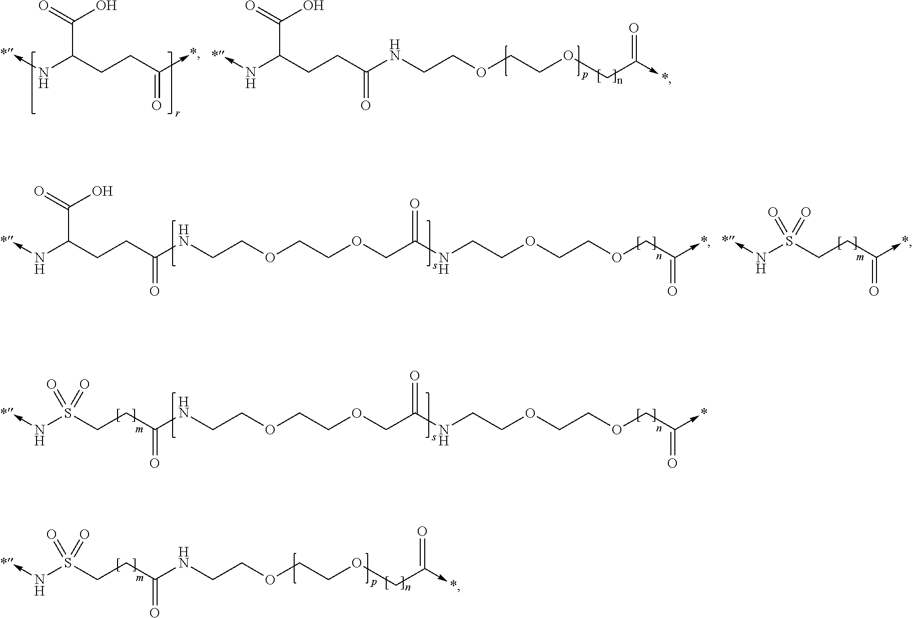
and 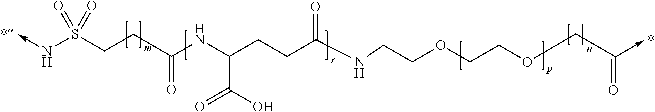
wherein m is 0, 1, 2 or 3; n is 1, 2 or 3; s is 0, 1, 2 or 3; p is 1, 2, 3 or 4 and; r is 1, 2 or 3; and
X is the attachment anchor to the Leptin compound and is selected from the group consisting of: 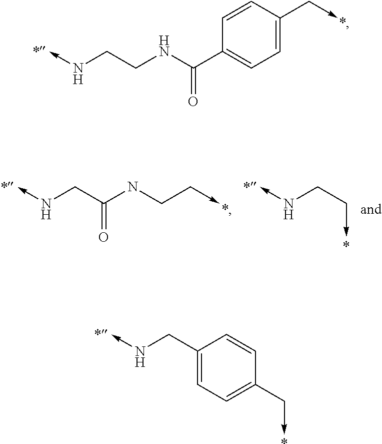
wherein “*” indicates the point of a moiety which is oriented towards the Leptin compound and “*″” indicates the point of a moiety which is oriented towards Z.

5. The compound according to claim 1, wherein the Z—Y—X-moiety is attached to the Leptin compound by alkylation chemistry.

6. The compound according to claim 1, wherein the Leptin compound is an analogue of Leptin.

7. The compound according to claim 1, wherein Z comprises a fatty acid or fatty diacid.

8. The compound according to claim 1, wherein Z comprises an alpha and omega carboxy group.

9. The compound according to claim 7, wherein Z comprises a fatty acid or fatty diacid with 12-22 carbon atoms.

10. The compound according to claim 7, wherein Z comprises a fatty acid or fatty diacid with 16-20 carbon atoms.

11. The compound according to claim 1, wherein said compound is

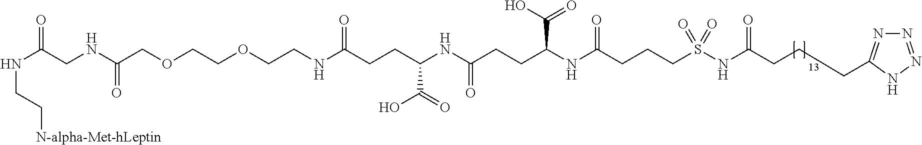

12. The compound according to claim 1, wherein said compound is

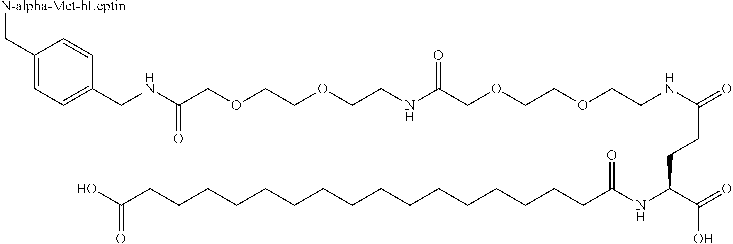

13. (canceled)

14. (canceled)

15. A composition comprising a compound as defined in claim 1 and a pharmaceutically acceptable excipients.

16. The compound according to claim 6, wherein the analogue of Leptin-is an analogue of rat Leptin.

17. The compound according to claim 6, wherein the analogue of Leptin-is an analogue of human Leptin.

18. The composition of claim 15 further comprising an anti-obesity agent or anti-diabetic agent.

19. The composition of claim 18, wherein the anti-diabetic agent comprises pramlintide.

20. A method of treating obesity comprising administering the compound of claim 1 to a patient in need thereof.
